(19) United States
(12) Patent Application Publication
(10) Pub. No.: US 2010/0221183 A1
(43) Pub. Date: Sep. 2, 2010

(54) PEPTIDES FOR TREATING AND DIAGNOSING CANCERS AND METHODS FOR USING THE SAME

(76) Inventor: Shayne Squires, Oxford, MA (US)

Correspondence Address: CLARK & ELBING LLP, 101 FEDERAL STREET, BOSTON, MA, 02110, US

(21) Appl. No.: 12/445,266

(22) PCT Filed: Oct. 10, 2007
(86) PCT No.: PCT/US07/81009

§ 371 Date: Oct. 23, 2009

Related U.S. Application Data
(60) Provisional application No. 60/850,542, filed on Oct. 10, 2006.

(51) Int. Cl. A61K 49/14; A61K 39/395; A61K 31/7088; C12P 21/02; G01N 33/574; C07K 16/18; C12Q 1/70

(52) U.S. Cl. 424/9.1; 424/178.1; 514/44 R; 435/69.6; 435/7.23; 530/387.9; 530/391.3; 530/391.7; 435/5

(57) ABSTRACT

Disclosed are peptides, polypeptides, antibodies, small molecules, and methods for their use for imaging a neoplasm and for treating cancer in a mammal. The methods include administering one or more of the agents of the invention to a mammal, e.g., a human; the peptides, polypeptides, antibodies, and small molecules, which specifically bind to neoplastic cells, can be labeled with a radioactive label or a therapeutic label, e.g., a cytotoxic agent.

### FIELD OF THE INVENTION

This invention relates to cancer therapeutics and diagnostic methods.

### BACKGROUND OF THE INVENTION

In 2002, deaths due to cancer represented 22.8% of deaths overall in the United States, ranking as the second leading cause of death. The four types of cancer that most commonly result in death are cancer of the lung or bronchus, breast cancer, colon or rectal cancer, and cancer of the prostate. Estimates for the year 2005 predict that lung cancer will account for 31% of cancer deaths in men and 27% of cancer deaths in women. The second leading cause of cancer deaths in women, breast cancer, is predicted to account for 15% of cancer deaths in women, while prostate cancer will likely represent 10% of cancer deaths in men. Colorectal cancer is expected to comprise 10% of cancer related deaths in men and women.

### Cancer Diagnosis

Each of the fatal cancers mentioned above is potentially curable if detected before metastasis has occurred. Surgical resection with the possible addition of radiation therapy or adjuvant chemotherapy remains the main therapeutic approach in appropriate cases. Early detection is sometimes accomplished through routine screening in a patient population at appropriately elevated risk. Screening procedures include colonoscopy and mammography for the early detection of colon and breast cancer respectively. Mammography remains an imperfect test because of the difficulty of detecting lesions in women with dense breasts. Prostate cancer screening consists of digital rectal examination (DRE) and measurement of blood prostate specific antigen (PSA) level. The sensitivity of DRE alone is only about 50%; the yield of combining DRE and PSA level evaluation is better, but whether early detection of prostate cancer saves lives remains a matter of controversy. Routine chest X rays to screen for lung cancer have not been definitively demonstrated to be effective. Current research focuses on whether CT scans of the chest might serve as an effective screening tool for the early detection of lung cancer, but the relatively high rate of incidental findings that must be followed up with subsequent testing present the challenge of cost effectiveness. 2-[¹⁸F] fluoro-2-deoxy-D-glucose (FDG) and positron emission tomography (PET) scanning (FDG-PET) scanning is sensitive for detecting primary malignancy in lung and breast but only if the lesion is more than 2 cm in size in the breast and more than 1 cm in lung. It has poor sensitivity and specificity for detecting primary prostate cancer. Because the FDG activity in the colon can be elevated normally, PET is a poor choice for screening for primary colon cancer. Given the current limitations of diagnostic testing, history and physical examination of patients remain the most important aspects of cancer diagnosis.

Once cancer has been diagnosed; the stage of the cancer must be determined. This consists of determining the size and extent of the tumor, whether it has invaded nearby structures, and whether it has spread to lymph nodes or more distant sites in the body. Whether a given treatment approach is effective depends upon the stage of the cancer at diagnosis. If the detection of cancer occurs at a relatively early stage, the patient can be treated with the intent to cure and surgical resection is usually essential. For more advanced stage cancers, treatment is administered to optimize the patient's quality of life and surgery will often cause morbidity without leading to a cure.

Scintigraphic studies have been well established as effective diagnostic tools in cancer staging. Bone scanning using diphosphonate compounds labeled with 99m-technetium have traditionally been used to detect osseous metastases in lung, breast, prostate, colorectal, and a variety of other cancers. The sensitivity of bone scans in this setting is generally accepted as 80-90% or higher, but these rates are largely extrapolated from small studies confined to one type of cancer. The specificity of bone scans alone is relatively poor and must be augmented by clinical information and supplemental radiography. FDG-PET scanning has emerged as a valuable tool in cancer staging. Its sensitivity for the detection of osseous metastases is nearly equal to that of bone scanning, but its specificity is much higher. Additionally, PET scanning can detect metastases in soft tissue, including metastases to lymph nodes, liver, adrenals, lung, and chest wall. Unfortunately PET scanning is not universally available in the United States. The expense associated with having and operating a PET imaging center requires such centers to exist in areas of higher population density. Many areas of the United States rely upon mobile PET units where the PET scanner is periodically transported to a community in the back of a truck. Some patients from rural areas have to travel for hours and pay for lodging to receive a PET scan. More traditional gamma cameras with Single-Photon Emission Computed Tomography (SPECT) capability are cheaper and easier to own and operate than PET scanners and consequently are more universally available. A single photon emitting radiotracer that enables detection and staging of cancer with a sensitivity and specificity similar to FDG could be imaged using a SPECT gamma camera and would be a great benefit to communities that cannot afford PET centers. Clearly this would not only benefit rural and semi-rural communities in the United States, but would also benefit regions without PET centers around the world. This could be accomplished by using molecules that specifically bind to cancer cells with relatively high affinity and which can be labeled with single photon emitters, such as 99m-technetium; a need for such molecules currently exists.

### Cancer Treatment

Current treatments for cancer include radiation, chemical, and biological therapies. Depending on the type, stage, location, and health of the patient, one or more of these types of cancer therapies may be employed following diagnosis. The role of biological therapies in the treatment of cancer is perhaps the fastest-developing area of cancer therapeutic research. Another developing line of therapeutics for cancer treatment involve synthetic small molecule drugs that arrest cancer growth and metastasis. The advantages and deficiencies of both biological and small molecule drug therapies for the treatment of cancer are discussed below.

Biological therapy for cancer typically involves eliciting anti-cancer immune responses in a patient that has cancer. This is a difficult proposition, as cancer cells, despite having a proliferative disorder, are largely recognized as “self” by the patient's immune system. Several strategies have evolved over the last two decades that utilize biological agents to fight cancer. Cytokines such as interleukin 2 (IL-2) and interferon alpha (IFN-α) are used against a broad spectrum of cancers, largely by stimulating the patient's immune system. These therapies can cause severe side effects, and do little to focus the immune response specifically against cancer cells. Alternatively, monoclonal antibodies, such as alemtuzumab (Campath) and rituximab (Rituxan) to have proven to be effective adjunct therapies to radiation and chemotherapy when used against certain types of cancer (e.g., B cell lymphocytic leukemia), although these and other monoclonal antibodies target cell types associated with the cancer, not solely cancerous cells. The partial or complete loss of entire cell populations or lineages (e.g., the loss of all lymphocytes when alemtuzumab is administered to a patient) is a troublesome and limiting side effect of many of these therapeutics. Only recently have monoclonal antibodies that specifically target cancerous cells, via tumor-specific antigens (TSA; e.g., trastuzumab, Herceptin) become a part of the clinician's arsenal against cancer. Biological therapies, including peptides, polypeptides, and antibodies that can discriminate between healthy and cancerous cells will provide significant treatment benefits.

Small molecule drugs that bind and inhibit cancerous cells represent another emerging therapeutic option for the treatment of cancer. Drugs such as erlotinib and gefitinib (EGFR tyrosine kinase inhibitors indicated for the treatment of non small cell lung cancer) are members of the first generation of small molecule agents that specifically target and inhibit cancer cells. Using computer modeling to design optimal binding and inhibition characteristics, research and development of anti-cancer small molecule drugs has dramatically increased in the last decade, with a number of products recently emerging on the market. Future efforts to identify and synthesize small molecule agents hold great promise for effective and targeted cancer therapy.

### SUMMARY OF THE INVENTION

This invention features compositions and methods for the diagnosis and staging of cancer, for monitoring a patient's responsiveness to cancer therapy, and for treating a patient with cancer. The invention is based upon the discovery of peptides, polypeptides, antibodies, and small molecules (i.e., agents of the invention) which bind with higher affinity to cancer cells than normal cells. Compositions and methods of the invention feature the diagnostic and therapeutic use of a panel of binding sequences, set forth in SEQ ID NOs:1-14, that facilitate binding of labeled or unlabeled agents of the invention specifically to cancer cells. In another embodiment of the invention, the agents of the invention bind to an epitope derived from the heat shock protein 70 (HSP70) protein, the sequence of which is set forth in SEQ ID NO:15. In particular embodiments, the peptides, polypeptides, antibodies, and small molecules disclosed in this invention bind with higher affinity to lung cancer, colon cancer, breast cancer, and prostate cancer cells than to noncancerous tissue.

In one aspect, the invention features agents (e.g., peptides, polypeptides, antibodies, and small molecules) that bind to heat shock protein 70 (HSP70) protein family members (e.g., HSP70, HSC71, GRP78, and mortalin) in their native, non-denatured conformations. In an embodiment, the agents are capable of binding to a cancer cell (e.g., a human neoplastic cell). In a preferred embodiment, the agents bind with greater affinity to cancer cells than to normal cells (e.g., non-cancerous cells). In another embodiment, the agents contain a binding domain that includes an amino acid sequence having at least 90% sequence identity to one or more of the sequences set forth in SEQ ID NOs: 1-14. In a preferred embodiment, the agents of the invention are antibodies (e.g., diabodies, bi-specific antibodies, Fab fragments, F(ab′)2 molecules, single chain Fv (scFv) molecules, tandem scFv molecules, monoclonal antibodies (mAbs), polyclonal antibodies, and antibody fusion proteins). In yet another embodiment, the antibodies may be humanized, chimeric, recombinant, synthetic, or naturally-derived, and may have one of the isotypes selected from IgG, IgA, IgM, IgD, or IgE. In other embodiments, the agents of the invention are coupled (e.g., directly (e.g., with or without a linker moiety) via a covalent bond, or indirectly via a charge interaction (e.g., via ionic, hydrophobic, hydrogen bonding, or Van der Waals forces)) to a detectable label (e.g., a radioactive label (e.g., technetium-99m, iodine-123, iodine-131, or indium-111), fluorescent label (e.g., a fluorophore), enzymatic label, heavy metal (e.g., a relaxivity metal); colorimetric label, or magnetic resonance imaging label), therapeutic or cytotoxic agent, or chelating agent. In a preferred embodiment, the agents of the invention contain an amino acid sequence having at least 80%, preferably 90%, more preferably 95%, and most preferably 99% or 100% sequence identity to one or more of the amino acid sequences set forth in SEQ ID NOs: 1-14. In another embodiment, the agents of the invention include a binding domain that contains the amino acid sequence DYWDTSWPLLLF (SEQ ID NO:11). Preferably, the agents of the invention target cancerous tissue (e.g. breast cancer, prostate cancer, colon cancer, or lung cancer). Another embodiment includes agents of the invention modified by PEGylation, cross-linking, or other chemical modifications. In a preferred embodiment, the agents of the invention are formulated with a pharmaceutically acceptable carrier or excipient.

A second aspect of the invention features methods of using the agents (e.g., peptides, polypeptides, antibodies, and small molecules) of the first aspect of the invention to image or diagnose cancer. In an embodiment of the invention, the agents are used to image a neoplasm or a region containing a neoplastic cell in a human patient or a sample thereof, in vivo or in vitro. In another embodiment, the methods are used to detect a neoplasm in a mammal in vivo or in a biological sample from the patient in an in vitro method. Preferably, these methods involve: (a) providing a detectably-labeled agent of the invention (e.g., peptide, polypeptide, antibody, or small molecule) having one or more binding domains containing one or more of the sequences set forth in SEQ ID NOs: 1-14 (or a molecule having at least 80%, preferably 90%, more preferably 95%, and most preferably 99% or 100% sequence identity to the amino acid sequences set forth in SEQ ID NOs: 1-14); (b) administering the agent to the patient (e.g., via intravenous injection) or contacting a biological sample taken from the patient; and (c) allowing the agent to bind to cancerous tissue and allowing unbound agents to be cleared from the body or sample; and (d) obtaining an image of, or detecting, the neoplasm or the region containing the neoplastic cell, or diagnosing cancer in the patient by detecting binding of the labeled agent to a cancer cell. In a preferred embodiment of the invention, the image so obtained may include the entire body in order to show the location of a primary cancer within the body or to detect the presence and distribution of cancer metastases throughout the body. Alternatively, the image may only include a region of the body (e.g., the head and neck, the thorax, the chest, or the abdomen) to better define the size or extent of neoplastic tissue within a region of the body. Preferably, the image is obtained using gamma scintigraphy.

In a third aspect, the invention features methods of using agents of the invention (e.g., peptides, polypeptides, antibodies, and small molecules) to treat cancer (e.g., a neoplasm) in a mammal in vivo. A preferable embodiment includes: (a) administering a diagnostically effective amount of a detectably-labeled molecule wherein said molecule is one of the peptides having the sequence set forth in SEQ ID NOs: 1-14 (or a molecule having at least 80%, preferably 90%, more preferably 95%, and most preferably 99% or 100% sequence identity to the peptide sequences set forth in SEQ ID NOs: 1-14); and (b) detecting the presence of the detectable label of the molecule bound to a tissue of the mammal, where an amount of label above background levels is indicative of the presence of the neoplasm in the mammal. In preferred embodiments, the method involves a human patient suspected of having a breast cancer, and the tissue is breast tissue. In other preferred embodiments, the method involves a human patient suspected of having a prostate cancer, and the tissue is prostate tissue. In other preferred embodiments, the method involves a human patient suspected of having a colon cancer, and the tissue is colon tissue. In other preferred embodiments, the method involves a human patient suspected of having a lung or bronchus cancer, and the tissue is lung or bronchus tissue. Preferably, the detectably labeled peptide is linked to a radionuclide (e.g., technetium-99m) and the detection step is accomplished by radioimaging (e.g., gamma scintigraphy).

In a fourth aspect, the invention features use of the peptides of the first aspect of the invention (e.g., one or more of the peptides having at least 80%, preferably 85%, 90%, or 95%, and more preferably 99% or 100% sequence identity to SEQ ID NOs:1-14) in a method for preparing antibodies with affinity to cancer cells. The antibodies can be produced recombinantly, and can be formulated for diagnostic or therapeutic use in the detection or treatment, respectively, of a cancer (e.g., a neoplasm) in a mammal in vivo. Antibodies according to the present invention have one or more binding domains capable of binding to a heat shock protein 70 (HSP70) protein family members (e.g., HSP70, HSC71; GRP78 and mortalin) in their native, non-denatured conformation. In preferred embodiments, the antibodies according to the present invention have one or more binding domains capable of binding to an epitope of the HSP70 protein having at least 90% sequence identity, preferably 95%, and most preferably 99% or 100% sequence identity to the sequence set forth in SEQ ID NO:15 (GIPPAPRGVPQIEVTF; amino acids 463-478 of HSP70). In preferred embodiments, at least one of the binding domains of the antibody of the present invention has an amino acid sequence selected from one or more of the sequences set forth in SEQ ID NOs:1-14. In another preferred embodiment, the binding domain of the antibody of the present invention includes the amino acid sequence DYWDTSWPLLLF (SEQ ID NO:11). In preferred embodiments, antibodies of the invention are recombinant, chimeric, humanized, synthetic, or naturally-derived antibodies. In other preferred embodiments, antibodies of the invention may be diabodies, bi-specific antibodies, Fab fragments, F(ab′)2 molecules, single chain Fv (scFv) molecules, tandem scFv molecules, monoclonal antibodies (mAbs), polyclonal antibodies, and antibody fusion proteins. Antibodies of the invention may also be any one of the isotypes selected from IgG, IgA, IgM, IgD, or IgE.

A fifth aspect of the present invention features methods for treating neoplastic conditions, such as treating, stabilizing, inhibiting or reducing the number or malignancy of cancer cells or treating, stabilizing, inhibiting or reducing the size of tumors, using antibodies of the present invention. In a preferred embodiment, the method includes administering a therapeutically-effective amount of an antibody of the invention, in which the antibody contains at least one binding domain having an amino acid sequence set forth in SEQ ID NOs: 1-14 (or an antibody having at least 80%, preferably 90%, more preferably 95%, and most preferably 99% or 100% sequence identity to the amino acid sequences set forth in SEQ ID NOs: 1-14). The method also includes administering a therapeutically-effective amount of an antibody that contains a binding domain that specifically binds an amino acid sequence with at least 90% sequence identity, preferably 95%, and more preferably 99% or 100% sequence identity to the sequence set forth in SEQ ID NO:15. In preferred embodiments, the method involves a human patient having a breast cancer, prostate to cancer, colon cancer, or lung or bronchus cancer. Preferably, the antibody is linked to a detectable label, cytotoxic or therapeutic agent, or a chelating agent directly (e.g., via a covalent bond or a linker moiety, or indirectly via a charge interaction).

A sixth aspect of the present invention features methods to determine whether a small molecule is capable of binding to the amino acid sequence set forth in SEQ ID NO:15, or an amino acid sequence with at least 90% identity to SEQ ID NO:15, with high affinity. This method also includes the screening of candidate small molecules that can preferentially bind cancer cells but not non-cancerous cells.

In a seventh aspect, a kit is provided for use in diagnostic or therapeutic embodiments of the invention. The kit includes an agent of the invention (e.g., a peptide, polypeptide, antibody, or small molecule) having the sequence set forth in SEQ ID NOs: 1-14 (or at least 80%, preferably 90%, more preferably 95%, and most preferably 99% or 100% sequence identity to the peptide sequences set forth in SEQ ID NOs: 1-14); and a detectable label, therapeutic agent, chelating agent, or a linker moiety. In a preferred embodiment, each component of the kit (a peptide, polypeptide or antibody and a detectable label, therapeutic agent, chelating agent, or linker moiety) is separately packaged in the kit. In another preferred embodiment, the kit includes a predetermined amount of the agent and the detectable label, therapeutic agent, chelating agent, or linker moiety (e.g., an amount sufficient for diagnosing or treating cancer in a subject). The agent and the detectable label, therapeutic agent, chelating agent, or linker moiety can be lyophilized to enable long-term storage. The peptide, polypeptide, or antibody and detectable label, therapeutic agent, chelating agent, or linker moiety can be sealed in a sterilized container. The kit preferably includes instructions for using the kit and its contents. For example, prior to use, Tc-99m-pertechnetate can be eluted from a Tc/Mo generator commonly present in nuclear medicine facilities and hospital radiopharmacies with isotonic sterile saline, and the peptide provided in the kit would be combined with Tc-99m-pertechnetate in the presence of the reducing agent to reduce a selected quantity of Tc-99m pertechnetate, and thereby, obtain the desired Tc-99m-peptide conjugates. Examples of reducing agents include but are not limited to stannous chloride and sodium dithionite. Examples of metal chelating agents include but are not limited to ininocarboxylic and polyaminopolycarboxylic reactive groups, diethylenetriaminepentaacetic acid (DTPA), and 1,4,7,10-tetraazacyclododecane-1,4,7,10-tetraacetic acid (DOTA).

The term “about” is used herein to mean a value that is ±10% of the recited value.

By “administration” or “administering” is meant a method of giving a dosage of a composition of the invention (e.g., a peptide, polypeptide, antibody, or small molecule) to a mammal (e.g., a human), where the method is, e.g., topical, oral, intravenous, intraperitoneal, or intramuscular. The preferred method of administration can vary depending on various factors, e.g., the components of the pharmaceutical composition, site of the potential or actual disease (e.g., the location of lung, breast, colon, or prostate cancer) and the severity of disease.

By “analog” is meant a molecule that differs from, but is structurally, functionally, and/or chemically related to the reference molecule. The analog may retain the essential properties, functions, or structures of the reference molecule. Most preferably, the analog retains at least one biological function of the reference molecule. Generally, differences are limited so that the structure or sequence of the reference molecule and the analog are similar overall. A peptide or polypeptide analog and its reference peptide or polypeptide may differ in amino acid sequence by one or more substitutions, additions, and/or deletions, in any combination. A substituted or inserted amino acid residue may or may not be a naturally occurring amino acid. An analog of a peptide or polypeptide may be naturally occurring such as an allelic variant, or it may be a variant that is not known to occur naturally. Non-naturally occurring analogs of peptides or polypeptides may be made by direct synthesis, by modification, or by mutagenesis techniques.

By “antibody” is meant all or a portion of a recombinant, synthetic, or natural antibody that includes one or more of the amino acid sequences set forth in SEQ ID NOs:1-14 (or a sequence having at least 90%, 95%, or 99% sequence identity to one or more of SEQ ID NOs:1-14 (i.e., at least one of the amino acid residues set forth in any of SEQ ID NOs:1-14 can be substituted with any other amino acid or can be to deleted)), which is present in a complementarity determining region. The term “antibody” encompasses any polypeptide capable of binding with specificity to an HSP70 protein, including e.g., complete, intact antibodies, chimeric antibodies, diabodies, bi-specific antibodies, Fab fragments, F(ab′)₂ molecules, single chain Fv (scFv) molecules, tandem scFv molecules, as well as fusion proteins that include the binding sequences set forth in SEQ ID NOs:1-14. Complete, intact antibodies include monoclonal antibodies such as murine monoclonal antibodies (mAb), polyclonal antibodies, chimeric antibodies, humanized antibodies and human antibodies. An antibody of the invention can be engineered to include polypeptide sequences from two or more mammalian species or from a single mammalian species. The antibody can also include synthetically derived sequences. In general, an antibody of the invention includes structural region sequences derived from a human antibody. The antibody of the invention can also be “humanized,” that is, it includes non-human residues in one or more regions, e.g., within one or more CDRs of the variable region. The antibodies of the invention provide increased epitope specificity and affinity to HSP70 proteins, and have increased systemic half-life.

In general, chimeric antibodies of the invention consist of binding sequences recombinantly derived from a non-human mammal (e.g., a mouse, rabbit, or goat) with the constant region sequence derived from a human antibody.

The production of antibodies and the protein structures of complete, intact antibodies, and antibody fragments such as Fab fragments, scFv fragments, and F(ab)₂ fragments and the organization of the genetic sequences that encode such molecules, are well known and are described, for example, in Harlow et al., ANTIBODIES: A LABORATORY MANUAL, Cold Spring Harbor Laboratory, Cold Spring Harbor, N.Y. (1988) and Harlow et al., USING ANTIBODIES: A LABORATORY MANUAL, Cold Spring Harbor Press, 1999, which are herein incorporated by reference in their entirety.

By “chelating agent” is meant a molecule that forms multiple chemical bonds with a single metal atom. Prior to forming the bonds, the chelating agent has more than one pair of unshared electrons. The bonds are formed by sharing pairs of electrons with the metal atom. Chelating agents include, for example, an iminodicarboxylic group or a polyaminopolycarboxylic group. Chelating agents may be attached to an agent of the invention (e.g., a peptide, polypeptide, antibody, or small molecule), using the methods generally described in Liu et al., Bioconjugate Chew. 12(4):653, 2001; Alter et al., U.S. Pat. No. 5,753,627; and PCT Publication No. WO 91/01144; each of which is hereby incorporated by reference. An agent of the invention may be complexed, through its attached chelating agent, to a detectable label, thereby resulting in an agent that is indirectly labeled. Similarly, cytotoxic or therapeutic agents may also be attached' via a chelating group to an agent of the invention.

By “complementarity determining region” or “CDR” is meant an amino acid sequence, or a nucleic acid sequence encoding the amino acid sequence, of an antibody which is the hypervariable region that enables binding of the antibody to a specific epitope (e.g., Kabat et al.; Sequences of Proteins of ImmunologicarInterest, 4th Ed., U.S. Department of Health and Human Services, National Institutes of Health (1987)). In a complete antibody, there are three heavy chain and three light chain CDRs (or CDR regions) in the variable region of the antibody. The variable region(s) which forms the antibody binding site, is aligned by a relatively conserved framework region (FR) that joins the variable region(s). CDR and FR residues are delineated according to the standard sequence definition of Kabat et al. (Sequences of Proteins of Immunological Interest (National Institutes of Health, Bethesda, Md. (1987) and (1991).

By “coupled” is meant the characteristic of a first molecule being joined to a second molecule by a covalent bond or through noncovalent intermolecular attraction.

By “cytotoxic agent” is meant any naturally-occurring, modified, or synthetic compound that is toxic to tumor cells. Such agents are useful in the treatment of neoplasms, and in the treatment of other symptoms or diseases characterized by cell proliferation or a hyperactive cell population. Cytotoxic agents include, but are not limited to, alkylating agents, antibiotics, antimetabolites, tubulin inhibitors, topoisomerase I and II inhibitors, hormonal agonists or antagonists, or immunomodulators. Cytotoxic agents may be cytotoxic when activated by light or infrared (Photofrin, IR dyes; Nat Biotechnol 19(4):327-331 (2001)), may operate through other mechanistic pathways, or be supplementary potentiating agents.

By “detectable label” is meant any type of label which, when attached to an agent of the invention (e.g., a peptide, polypeptide, antibody, or small molecule) renders the agent detectable. A detectable label may be toxic or non-toxic, and may have one or more of the following attributes, without restriction: fluorescence (Kiefer et al., WO 9740055), color, toxicity (e.g., radioactivity, e.g., a γ-emitting radionuclide, Auger-emitting radionuclide, β-emitting radionuclide, an α-emitting radionuclide, or a positron-emitting radionuclide), radiosensitivity, or photosensitivity. A detectable label may be directly attached to an agent of the invention (e.g., to a residue of a peptide, polypeptide, or antibody, or by a chemical bond to a small molecule) or indirectly attached to an agent of the invention, for example, by being complexed with a chelating group that is attached (e.g., via a covalent bond or indirectly linked) to the agent. A detectable label may be indirectly attached to an agent of the invention by the ability of the label to be specifically bound by a second molecule. One example of this type of an indirectly attached label is a biotin label that can be specifically bound by the second molecule, streptavidin. The second molecule may also be linked to a moiety that allows neutron capture (e.g., a boron cage as described in, for example, Kahl et al., Proc Natl Acad Sci USA 87:7265-7269 (1990)).

A detectable label may also be a metal ion from heavy elements or rare earth ions, such as Gd³⁺, Fe³⁺, Mn³⁺, or Cr²⁺ (e.g., Invest Radiol 33(10):752-761, 1998). Preferred radioactive detectable labels include radioactive iodine labels (e.g., ¹²²I, ¹²³I, ¹²⁴I, ¹²⁵I, or ¹³¹I) that are capable of being coupled to each D- or L-Tyr or D- or L-4-amino-Phe residue present in the analogs of the invention. Preferred non-radioactive detectable labels include the many known dyes that are capable of being coupled to NH₂-terminal amino acid residues.

Preferred examples of detectable labels that may be toxic to cells include ricin, diptheria toxin, and radioactive detectable labels (e.g., 122I, ¹²³I, ¹²⁴I, ¹²⁵₁, ¹³¹I, ¹⁷⁷Lu, ⁶⁴Cu, ⁶⁷Cu, ¹⁵³Sm, ¹⁶⁶Ho, ¹⁸⁶Re, ¹⁸⁸Re, ²¹¹At, ²¹²Bi, ²²⁵Ac, ⁶⁷Ga, ⁶⁸Ga, ⁷⁵Br, ⁷⁶Br, ⁷⁷Br, ^{117m}Sn, ⁴⁷Sc, ¹⁰⁹Pd, ⁸⁹Sr, ¹⁵⁹Gd, ¹⁴⁹Pm, ¹⁴²Pr, ¹¹¹Ag, ¹⁶⁵Dy, ²¹³Bi, ¹¹¹In, ^{114m}In, ²⁰¹Ti, ^{195m}Pt, ¹⁹³Pt, ⁸⁶Y and ⁹⁰Y). These compounds, and others described herein may be directly or indirectly attached to an agent of the invention (e.g., a peptide, polypeptide, antibody, or small molecule) or its analogs. A toxic detectable label may also be a chemotherapeutic agent (e.g., camptothecins, homocamptothecins, 5-fluorouracil or adriamycin), or may be a radiosensitizing agent (e.g., Taxol, gemcitabine, fluoropyrimidine, metronitozil, or the deoxycytidine analog 2′,2′ difluoro-2′-deoxycytidine (dFdCyd).

A detectable label, when coupled to an agent of the invention (e.g., a peptide, polypeptide, antibody, or small molecule) emits a signal that can be detected by a signal transducing machine. In some cases, the detectable label can emit a signal spontaneously, such as when the detectable label is a radionuclide. In other cases the detectable label emits a signal as a result of being stimulated by an external field such as when the detectable label is a relaxivity metal. Examples of signals include, without limitation, gamma rays, X-rays, visible light, infrared energy, and radiowaves. Examples of signal transducing machines include, without limitation, gamma cameras including SPECT/CT devices, PET scanners, fluorimeters, and Magnetic Resonance Imaging (MRI) machines.

By “diagnostically effective amount” is meant a dose of detectably-labeled agent of the invention (e.g., a peptide, polypeptide, antibody, or small molecule) which, when administered internally to a mammal, is quantitatively sufficient to be detected by a signal transducing machine external to the mammal (e.g., a gamma camera used in gamma scintigraphy), but which typically is quantitatively insufficient to produce a pharmacological effect.

By “epitope” is meant a region on an antigen molecule to which an antibody or a portion thereof binds specifically. The epitope can result from a three dimensional sequence formed from residues on different regions of a protein antigen molecule, which, in a native state, are closely apposed due to protein folding, or can result from a linear sequence of a protein or peptide in a denatured conformation. “Epitope” as used herein can also mean an epitope created by a peptide or hapten portion of an HSC70 protein family member (e.g., HSP70, GRP78, HSC71) and not a three dimensional epitope. Preferred epitopes are those wherein when bound to an immunogen (antibody, antibody fragment, or immunogenic fusion protein) results in inhibited or blocked cancer proliferation or metastasis.

“Heat shock protein” or “HSP” is meant a protein that is expressed in a cell that is exposed to cellular or environment stress (e.g., sudden elevations in temperature or glucose deprivation). The family of heat shock proteins includes HSP70 proteins, which participate in protein translocation across membranes. HSP70 proteins are generally increased in cancerous cells. Examples of HSP70 and HSP family members are described in, e.g., U.S. Pat. No. 5,627,039 and U.S. Patent Application Publication Nos. 20030211102 and 20060270622, which are incorporated by reference herein.

By “humanized antibody” means a type of chimeric antibody comprising a human framework region and one or more CDRs from a non-human (usually a mouse or rat) antibody. The non-human antibody providing the CDRs is called the “donor” and the human antibody providing the framework is called the “acceptor”. Constant regions need not be present, but if they are, they should be substantially identical to human antibody constant regions, i.e., at least about 85-90%, preferably about 95% or more identical. Hence, all parts of a humanized antibody, except possibly the CDRs, are substantially identical to corresponding parts of natural human antibody sequences.

By “imaging agent” is meant a composition of matter that, when administered to a living subject, such as a mammal (e.g., a human), allows the visualization of internal structures of the subject or allows measurements to be made with respect to the functioning of the subject's tissue or organs. Examples of imaging agents include, e.g., FDG, which can be directly or indirectly attached to an agent of the invention (e.g., a peptide, polypeptide, antibody, or small molecule).

By “linker moiety” is meant an amino acid sequence that couples an agent of the invention (e.g., a peptide, polypeptide, antibody, or small molecule) to a chelating agent.

By “neoplasm” is meant any tissue, or cell thereof, characterized by abnormal growth as a result of excessive cell division. The neoplasm can be benign or malignant. Examples of neoplasms include, without limitation, lune or bronchus carcinomas, colorectal carcinomas, breast carcinomas, and prostate carcinomas.

By “peptide” is meant an amino acid sequence that includes 5 or more amino acid residues. “Peptide” refers to both short chains, commonly referred to as peptides, oligopeptides, or oligomers, and to longer chains, up to about 100 residues in length, and can include cyclic or branched peptides joined to each other by peptide bonds or modified peptide bonds. Peptides may contain amino acids other than the 20 gene-encoded amino acids (i.e., non-naturally occurring amino acids), and linkages other than peptide bonds (e.g., peptoid of N-substituted glycine bonds). “Peptides” include amino acid sequences modified either by natural processes, or by chemical modification techniques which are well known in the art. Modifications may occur anywhere in a peptide sequence, including the peptide backbone, the amino acid side-chains, and the amino or carboxyl termini.

The notations used herein for the amino acid residues are those abbreviations commonly used in the art. The less common abbreviations Abu, Ava, β-Ala, hSer, Nle, Nva, Pal, Dab, and Dap stand for 2-amino-butyric acid, amino valeric acid, beta-aminopropionic homoserine, norleucine, norvaline, (2,3, or 4) 3-pyridyl-Ala, 1,4-diaminobutyric acid, and 1,3-diaminopropionic acid, respectively. In all aspects of the invention, it is noted that when amino acids are not designated as either D- or L-amino acids, the amino acid is either an L-amino acid or could be either a D- or L-amino acid.

By “reducing agent” is meant a chemical compound used to reduce another chemical compound by donating electrons, thereby becoming oxidized. Examples of reducing agents include but are not limited to lithium aluminum hydride (LiAlH₄), nascent hydrogen, sodium amalgam, sodium borohydride (NaBH₄), stannous ion, sulfite compounds, hydrazine (Wolff-Kishner reduction), Zinc-mercury amalgam (Zn(Hg)), diisobutylaluminum hydride (DIBAH), Lindlar catalyst, and oxalic acid (C2H2O4).

By “specifically binds” is meant that an agent of the invention (e.g., a peptide, polypeptide, antibody, or small molecule) recognizes and binds to a target (e.g., a neoplastic cell, such as a breast cancer cell, a prostate cancer cell, a colon cancer cell, or a lung cancer cell), but does not substantially recognize and bind to a non-target (e.g., non-neoplastic cells), whether the target is present in vivo or in an in vitro sample, e.g., a biological sample that includes, e.g., neoplastic cells. A desirable agent of the invention specifically binds to cancer cells, e.g., breast cancer cells, prostate cancer cells, colon cancer cells, or lung cancer cells. Preferably, an agent of the invention binds neoplastic cells with at least 2, 5, 10, 20, 100, or 1000 fold greater affinity than it binds to non-neoplastic cells. In addition, agents of the invention bind to the HSP70 epitope set forth in SEQ ID NO:15 with a dissociation constant less than 10⁻⁶M, more preferably less than 10⁻⁷M, 10⁻⁸M, 10⁻⁹M, 10⁻¹⁰M, 10⁻¹¹M, or 10⁻¹²M, and most preferably less than 10⁻¹³M, 10⁻¹⁴ M, or 10¹⁵M. By “substantial sequence identity” or “substantially identical” is meant a peptide or polypeptide (including but not limited to an antibody or antibody fragment sequence) exhibiting at least 50%, preferably 60%, 70%, 75%, or 80%, more preferably 85%, 90% or 95%, and most preferably 99% identity to a reference amino acid sequence. The length of the comparison sequence will generally be at least 5 amino acids, preferably at least 10 contiguous amino acids, more preferably at least 15, 20, 25, 30, 40, 50, 60, 80, 90, 100, 150, 200, 250, 300, or 350 contiguous amino acids, and most preferably the full-length amino acid sequence. Preferably, the sequence of the peptide of the invention is at least 40, 50, 60, 70, 80, 90, 95, or 99% identical to the reference sequence (e.g., one or more of the peptides set forth in SEQ ID NOs.: 1-14). Sequence identity is typically measured using BLAST® (Basic Local Alignment Search Tool) or BLAST®2 with the default parameters specified therein (e.g., Altschul et al., J Mol Biol 215:403-410 (1990); and Tatiana et al., FEMS Microbial Lett 174:247-250 (1999)). This software program matches similar sequences by assigning degrees of homology to various substitutions, deletions, and other modifications. Conservative substitutions typically include substitutions within the following groups: glycine, alanine, valine, isoleucine, leucine; aspartic acid, glutamic acid, asparagine, glutamine; serine, threonine; lysine, arginine; and phenylalanine, tyrosine.

By “therapeutic agent” is meant any compound known in the art that is used in the detection, diagnosis, or treatment of cancer. Such compounds may be naturally-occurring, modified, or synthetic. A therapeutic agent may be, for example, antineoplastic, including cytostatic and/or cytotoxic. Antineoplastic agents may be alkylating agents, antibiotics, antimetabolites, hormonal agonists or antagonists, tubulin inhibitors, topoisomerase I and II inhibitors, anti- or pro-apoptotic agents, or immunomodulators. Antineoplastic agents may operate through other mechanistic pathways, or antineoplastic agents may be supplementary potentiating agents.

By “therapeutically effective amount” is meant a dose of an agent of the invention (e.g., a peptide, polypeptide, antibody, or small molecule) which, when administered internally to a mammal, is sufficient to produce a pharmacological effect. For example, in the treatment of cancer or tumors, a “therapeutically effective amount” is an amount sufficient to produce a clinically significant effect, such as a stabilization or reduction in the size of a tumor or in the proliferation of cancer cells, or a statistically significant improvement in the survival of patients receiving a course of therapeutic treatment with an agent of the invention in accordance with the present methods of the invention, as compared to a control patient who has not received such a course of treatment.

By “treating, stabilizing, or inhibiting cancer” is meant causing a reduction in the size of a tumor or in the number of cancer cells, slowing or preventing an increase in the size of a tumor or cancer cell proliferation, increasing the disease-free survival time between the disappearance of a tumor or other cancer and its reappearance, preventing an initial or subsequent occurrence of a tumor or other cancer, or reducing an adverse symptom associated with a tumor or other cancer. In a desired embodiment, the percent of tumor or cancerous cells surviving the treatment is at least 20, 40, 60, 80, or 100% lower than the initial number of tumor or cancerous cells, as measured using any standard assay (e.g., caspase assays, TUNEL and DNA fragmentation assays, cell permeability assays, and Annexin V assays). Desirably, the decrease in the number of tumor or cancerous cells induced by administration of an agent of the invention is at least 2, 5, 10, 20, or 50-fold greater than the decrease in the number of non-tumor or non-cancerous cells. Desirably, the methods of the present invention result in a decrease of 20, 40, 60, 80, or 100% in the size of a tumor or in the number of cancerous cells, as determined using standard methods. Desirably, at least 20, 40, 60, 80, 90, or 95% of the treated subjects have a complete remission in which all evidence of the tumor or cancer disappears. Desirably, the tumor or cancer does not reappear or reappears after at least 5, 10, 15, or 20 years.

Other features and advantages of the invention will be apparent from the following description of the preferred embodiments thereof, and from the claims.

### BRIEF DESCRIPTION OF THE DRAWINGS

FIG. 1 is a chart showing an algorithm for panning phage libraries against different cancer cell types and fibroblasts to select phage clones which bind various types of cancer but not normal fibroblasts. The cyclical algorithm was repeated for multiple iterations.

FIG. 2 is a table showing consensus sequences for cancer binding phage peptides. After the final round of panning against cancer cells, the unamplified phage pool was analyzed for consensus sequences. Twenty-four clones were selected from the 12mer pool, and thirteen clones were selected from the 7mer pool.

FIGS. 3A-3C are micrographs of cellular internalization studies using peptide formulation PanF. Cancer cells (FIG. 3A) NCI-H460, (FIG. 3B) DU-145, and fibroblasts (FIG. 3C) CCD-1070Sk are incubated with fluorescein labeled peptide (green). Cell nuclei are visualized with DAPI (blue). Significant internalization is seen in cancer cells but not fibroblasts.

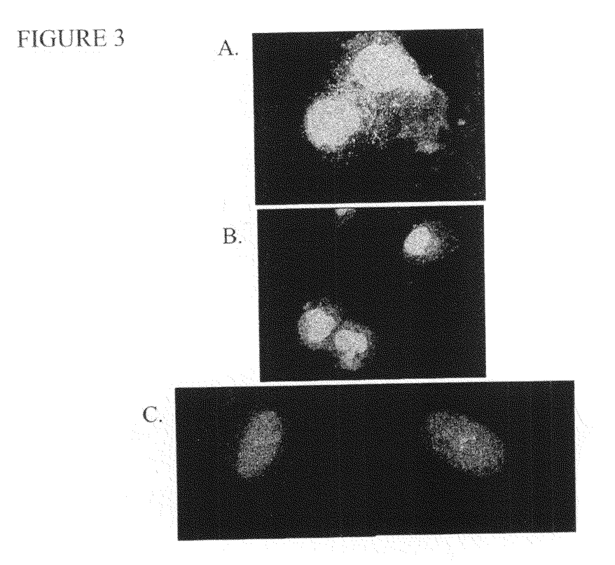

FIG. 4 is a graph showing percent cytotoxicity induced by incubating cells with PanL. Cell cultures consisting of prostate cancer, lung cancer, nonmalignant prostate epithelium, and fibroblasts were incubated in the presence of DYWDTSWPLLLFGGGS (KFAKFAK)₃ (PanL; SEQ ID NO:13). A dose dependent increase in cell death is seen in the case of tumor cells. Nonmalignant cells show a lower degree of susceptibility.

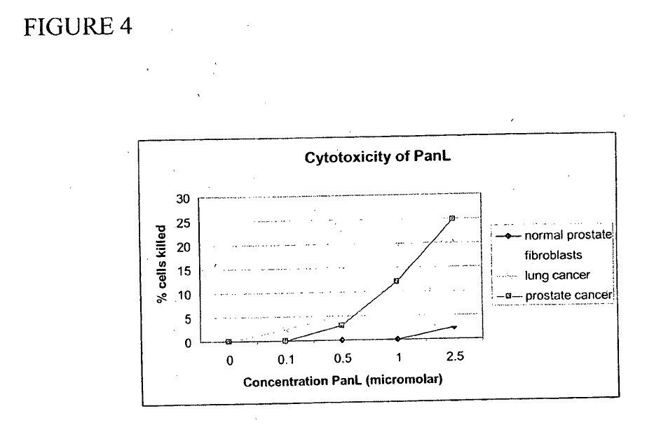

FIG. 5 is a graph showing the percentage of cells killed by cancer binding peptide (PanC) conjugated to ricin A subunit. The concentration of ricin conjugate is 1000 nM.

FIG. 6 is a photograph of polyacrylamide gel electrophoresis of eluate from affinity capture of cancer peptide binding targets was performed and stained with Coomassie blue. Lane 2 contains a ladder of molecular weight standards. Lane 5 shows a single prominent band corresponding to a mass of approximately 70 kD.

FIG. 7 is a graph showing the binding of radio-labeled PanC to the candidate epitope. This is tested by incubating epitope with 20 nM of labeled PanC. Increasing amounts of unlabeled PanC are added to the incubation mixture which blocks labeled PanC from binding.

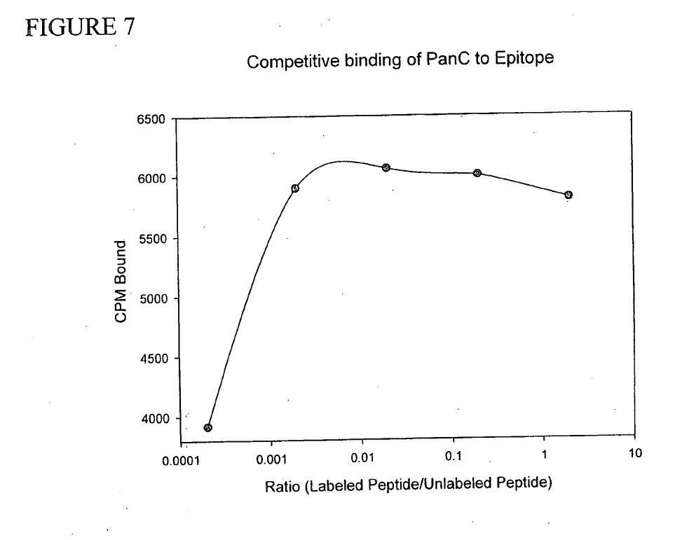

FIG. 8 is a graph showing that the Hsc71 epitope interferes with peptide binding to Hsp70. Radio-labeled PanC is added to Hsp70 coated wells and BSA coated wells (as a control) in the presence of various concentrations of epitope (GIPPAPRGVPQIEVTF; SEQ ID NO:15) from Hsc71. PanC concentration is constant. The epitope interferes with PanC binding to Hsp70 in a concentration dependent manner, but it suppresses binding of PanC to BSA approximately equally at all concentrations, consistent with specific binding competition between the epitope and Hsp70 but not between the epitope and BSA.

### DETAILED DESCRIPTION OF THE INVENTION

The invention features methods and compositions for the diagnosis and treatment of cancer. The inventor has determined that cancer cells express higher levels of certain membrane bound molecules, e.g., proteins in the heat shock protein 70 (HSP70) family, such as HSP70 (also known as HSP72), HSC70, (also known as HSP73), Grp78 (also known as BiP), mortalin, HSP60, and HSC71, than non-cancerous cells. As a result, these molecules represent valuable targets for directing therapeutic and diagnostic agents specifically to cancer cells, thereby facilitating detection of, and/or targeting of cytotoxic agents to, cancer cells, and eliminating or reducing toxicity to surrounding non-cancerous cells and tissues. Previous efforts to capitalize on the differential display of molecules on cancer cells have failed to identify useful epitopes that could be targeted by therapeutic or diagnostic agents. The compositions and methods of the present invention utilize an agent (e.g., a peptide, polypeptide, antibody, or small molecule) directed against cancer cells that differentially express an HSP70 protein family member. The agents can be used for diagnosis or detection of cancer cells, or can be used to induce apoptosis or necrosis in cancer cells, by modifying the agent to include a detectable label or a therapeutic/cytotoxic agent, respectively. The agents of the invention specifically bind the defined epitope GIPPAPRGVPQIEVTF (SEQ ID NO:15) or an epitope that has at least 90%, 95%, or 99% sequence identity to this defined epitope. The epitope is present in HSP70 family proteins (e.g., amino acids 463-478 of HSP70). In particular, agents of the invention bind this HSP70 epitope, either alone or when it is in the context of a polypeptide sequence (e.g., in the context of a polypeptide displayed on a cancer cell). Preferably, the agents of the invention bind the HSP70 epitope when it is displayed in its native conformation in an HSP70 protein on a cancer cell. Thus, denaturation of the HSP70 protein is not required for the agents of the invention to bind the defined epitope. Taken together, the methods and agents of the invention provide direct targeting of cancer therapeutic and diagnostic agents which will reduce bystander toxicity.

The agents of the invention also provide benefits to the field of medical cancer imaging. Imaging molecules can be conjugated to agents of the invention and used to obtain images that establish the presence of cancer cells in the body of a mammal administered the imaging agent. Alternatively, imaging molecules conjugated to agents of the invention can be contacted to a biological sample from the mammal, which is screened for binding of the imaging agent to cancer cells in the sample. The detection of a signal emitted from the imaging agent that is bound to the cancer cell via the agent of the invention confirms the presence of cancer cells in the mammal or a biological sample from the mammal. The imaging agents of the invention, when used in vivo, can be used to image the entire body. In addition, the imaging agents of the invention can be used to determine the stage of a patient's cancer, which can facilitate the identification of appropriate subsequent management. Furthermore, the imaging agents of the invention can be used to detect the response of cancerous tissue during and following therapy. Occasionally, previously cancerous masses can persist on radiographic imaging, e.g., CT scan, despite successful treatment. In this setting, the use of the imaging agents of the invention disclosed herein can be used to distinguish a successfully treated mass from one that is not successfully treated; the absence of emission of signal from a mass following the administration of the imaging agents disclosed in this invention indicates that the mass has been successfully treated. The present invention allows for imaging of tumors using PET and SPECT imaging, which are cheaper and more widely available.

### Preparation of Peptide or Polypeptide Agents of the Invention

Peptide and polypeptide agents of the invention capable of targeting cancer cells can be prepared by coupling using solid phase peptide synthesis (SPPS). As is well known in the art, the amino acids to be used as substrates to form the peptides are Fmoc-protected prior to incorporation into a peptide (see, e.g., Chan, W. C. and White, P. D., FMOC Solid Phase Peptide Synthesis, A Practical Approach, Oxford University Press, New York (2003); incorporated herein by reference in its entirety). The standard coupling techniques used to couple the amino acids in order to form the peptides and polypeptides disclosed in this invention are well known in the art (see, e.g., Chan and White, supra). For example, a polyamide-Rink resin can be prepared by loading a polyamide resin with Fmoc-Rink using chemical protocols well known in the art (see, e.g., Chan and White, supra). Using techniques well known in the art, the first amino acid in the peptide or polypeptide sequence is coupled to the resin after removing Fmoc from the N-terminal amine of the resin using piperidine. Once the coupling is complete, the resin is washed and Fmoc is removed from the coupled amino acid using piperidine. The resin is washed again, and the next amino acid in the sequence is coupled to the previously coupled amino acid using techniques well known in the art. This process is repeated using the necessary amino acids until the desired peptide or polypeptide is formed. Following the coupling of the final amino acid, the Fmoc group is removed using piperidine. The terminal amine can be left as a free amine, or it can be acetylated using techniques well known in the art (see, e.g., Chan and White, supra). The peptide or polypeptide can be cleaved from the resin using trifluoroacetic acid (TFA), triisopropylsilane, and water according to techniques known in the art (see, e.g., Chan and White, supra). The cleaved peptide or polypeptide is then separated from the residue by filtration. The TFA is typically evaporated to dryness followed by precipitation of the peptide or polypeptide with diethyl ether. Typically, the final peptide or polypeptide product is purified using HPLC according to techniques well known in the art (see, e.g., Chan and White, supra). Mass spectrometry is used to verify that the desired peptide or polypeptide is obtained. The peptides and polypeptides disclosed in this invention can be readily prepared by automated solid phase synthesis using any one of a number of well known, commercially available automated synthesizers, such as Applied Biosystems ABI 433A peptide synthesizer.

Peptide or polypeptide agents of the invention can also be isolated from a natural source, recombinantly produced, or synthesized by other techniques known in the art.

### Small Molecules of the Invention

The invention also features small molecules that serve diagnostic or therapeutic functions based on their ability to specifically bind cancer cells displaying HSP70 family proteins via the epitope set forth in SEQ ID NO:15 (or an epitope with at least 90%, 95%, or 99% sequence identity to this defined epitope). Small molecules of the invention can be labeled or fused to diagnostic or therapeutic linkers, markers, cytotoxic agents, or other embodiments of the invention to aid in the diagnosis or treatment of cancer.

### Methods of Screening Small Molecules for Binding to Hsp70 Proteins

The invention features methods for the high throughput screening (HTS) of candidate small molecule agents of the invention for ability to bind HSP70 family proteins, particularly the sequence set forth in SEQ ID NO:15. Candidate small molecules will also be screened for their ability to bind cancer cells, inhibit growth, or alter metastasis potential. In general, candidate small molecules must bind target sequences with a dissociation constant less than 10⁻⁶ M for further consideration as an agent of the invention.

Peptides, polypeptides, phages, or fusion molecules, or libraries thereof, encoding the epitope set forth in SEQ ID NO:15, or a sequence having at least 90% identity to SEQ ID NO:15, will be used HTS binding assays and methods. In general, fluorescence and luminescence based assays (e.g., ELISA, colorimetric assays) are used to measure binding affinities of candidate small molecules contacted against single or multiple target compounds that encode the epitope set forth in SEQ ID NO:15. Upon the identification of a candidate small molecule from a first screening process, it is necessary to further scrutinize the binding affinity and ability of the candidate by means of a second, different HTS assay. This could be accomplished, for example, by contacting the promising candidate small molecule with variants of the epitope set forth in SEQ ID NO:15 to more precisely determine the binding affinity of the molecule. A discussion of HTS methodologies is found in Verkman, “Drug discovery in academia,” Am. J. Physiol. Cell Physiol. 286, C465-C474 (2004) and Dove, “Screening for content—the evolution of high throughput,” Nat Biotechnol 21:859-864 (2003). Examples of HTS screening methods for the discovery of useful small molecule agents are found in, e.g., U.S. Pat. Nos. 7,279,286 and 7,276,346, and are incorporated by reference herein.

Candidate small molecules that have undergone HTS screening may be further modified to empirically improve binding affinities or cancer cell growth inhibition properties according to the design considerations discussed below.

### Small Molecule Design

Small molecules of the invention can also be generated according to the principles of rational design. Computer modeling technology allows visualization of the three-dimensional atomic structure of a selected molecule and the design of new compounds that will interact with HSP70 family proteins via the epitope set forth in SEQ ID NO:15 or other epitopes unique to HSP70 proteins expressed on cancer cells. The three-dimensional construct typically depends on data from x-ray crystallographic analyses or NMR imaging of the selected molecule or epitope. A computer graphics system enables prediction of how a candidate small molecule compound will bind to the target HSP70 family protein or epitope and allows experimental manipulation of the structures of the small molecule and target protein to perfect binding specificity. A prediction of what the molecule-protein interaction will be when small changes are made in one or both can be determined by using molecular mechanics software and computationally intensive computers. An example of a molecular modeling system described generally above includes the CHARMm and QUANTA programs (Polygen Corporation, Waltham, Mass.). CHARMm performs the energy minimization and molecular dynamics functions, while QUANTA performs the construction, graphic modeling and analysis of molecular structure. QUANTA allows interactive construction, modification, visualization, and analysis of the behavior of molecules that intact with each other. Another molecular modeling program that can be used to identify small molecules for use in the methods of the invention is DOCK (Kuntz Laboratory, UCSF).

The conformational and structural properties of HSP70 family proteins are known to those with skill in the art (see Bork et al., “An ATPase domain common to prokaryotic cell cycle proteins, sugar kinases, actin, and hsp70 heat shock proteins,” Proc Nall Acad Sci USA 89(16):7290-4 (1992), Bukau et al., “The Hsp70 and Hsp60 chaperone machines,” Cell 92(3):351-66 (1998), Misselwitz et al., “J proteins catalytically activate Hsp70 molecules to trap a wide range of peptide sequences,” Mol Cell 2(5):593-603 (1998), Osipiuk et al., “Structure of a new crystal form of human Hsp70 ATPase domain,” Acta Crystallogr D Biol Crystallogr. (Pt 5):1105-7 (1999), Sondermann et al., “Structure of a Bag/Hsc70 complex: convergent functional evolution of Hsp70 nucleotide exchange factors,” Science 291(5508):1553-7 (2001), and Tutar, “Key residues involved in Hsp70 regulatory activity and affect of co-chaperones on mechanism of action,” Protein Pept Lett 13(7):693-8 (2006). This knowledge can be used to design small molecules capable of binding to HSP70 proteins.

### Small Molecule Synthesis

Small molecules of the invention can be organic or inorganic compounds, and even nucleic acids. Specific binding to the targeted HSP70 family protein or epitope can be achieved by including chemical groups having the correct spatial location and charge in the small molecule. In a preferred embodiment, compounds are designed with hydrogen bond donor and acceptor sites arranged to be complementary to the targeted molecule or epitope. An agent is formed with chemical side groups ordered to yield the correct spatial arrangement of hydrogen bond acceptors and donors when the agent is in a specific conformation induced and stabilized by binding to the target molecule or epitope. Additional binding forces such as ionic bonds and Van der Waals interactions can also be considered when synthesizing a small molecule of the invention. The likelihood of forming the desired conformation can be refined and/or optimized using molecular computational programs.

Organic compounds can be designed to be rigid, or to present hydrogen bonding groups on edge or plane, which can interact with complementary sites. Rebek, Science 235, 1478-1484 (1987) and Rebek, et al., J Am Chem Soc 109, 2426-2431 (1987), have summarized these approaches and the mechanisms involved in binding of compounds to regions of proteins.

Synthetic methods can be used by one skilled in the art to make small molecules that interact with functional groups in the minor groove of HSP70 family proteins or epitopes.

### Preparation of Antibody Compositions of the Invention

The invention also provides antibody compositions that include one or more of the sequences set forth in SEQ ID NOs: 1-14. Additionally, the invention provides for antibodies that bind the epitope set forth in SEQ ID NO:15, preferably in its undenatured, native conformation. The antibodies of the invention may be recombinant (e.g., chimeric or humanized), synthetic, or natural antibodies. The invention features complete antibodies, diabodies, bi-specific antibodies, antibody fragments, Fab fragments, F(ab′)₂ molecules, single chain Fv (scFv) molecules, tandem scFv molecules, or antibody fusion proteins. Antibodies of the invention include the IgG, IgA, IgM, IgD, and IgE isotypes. Antibodies of the invention contain one or more CDR regions or binding peptides that bind to HSP70 family proteins at the epitope set forth in SEQ ID NO:15, or a sequence having 90%, 95%, or 99% sequence identity to this defined epitope. Antibodies of the invention bind the sequence set forth in SEQ ID NO:15, or a sequence with at least 90% sequence identity to SEQ ID NO:15, with a dissociation constant less than 10⁻⁶M, more preferably less than 10⁻⁷M, 10⁻⁸M, 10⁻⁹M, 10⁻¹⁹M, 10⁻¹¹M, or 10⁻¹²M, and most to preferably less than 10⁻¹³M, 10⁻¹⁴M, or 10⁻¹⁸M.

Many of the antibodies, or fragments thereof, described herein can undergo non-critical amino-acid substitutions, additions or deletions in both the variable and constant regions without loss of binding specificity or effector functions, or intolerable reduction of binding affinity (i.e., below about 10⁻⁷ M). Usually, an antibody incorporating such alterations exhibits substantial sequence identity to a reference antibody from which it is derived. Occasionally, a mutated antibody can be selected having the same specificity and increased affinity compared with a reference antibody from which it was derived. Phage-display technology offers powerful techniques for selecting such antibodies. See, e.g., Dower et al., WO 91/17271 McCafferty et al., WO 92/01047; and Huse, WO 92/06204, incorporated by reference herein.

### Antibody Fragments

In another embodiment of the invention, an agent of the invention is a fragment of an intact antibody described herein. Antibody fragments include separate variable heavy chains, variable light chains, Fab, Fab′, F(ab′)₂, Fabc, and Fv. Fragments can be produced by enzymatic or chemical separation of intact immunoglobulins. For example, a F(ab′)₂ fragment can be obtained from an IgG molecule by proteolytic digestion with pepsin at pH 3.0-3.5 using standard methods such as those described in Harlow and Lane, Antibodies: A Laboratory Manual, Cold Spring Harbor Pubs., N.Y. (1988). Fab fragments may be obtained from F(ab′)₂ fragments by limited reduction, or from whole antibody by digestion with papain in the presence of reducing agents. Fragments can also be produced by recombinant DNA techniques. Segments of nucleic acids encoding selected fragments are produced by digestion of full-length coding sequences with restriction enzymes, or by de novo synthesis. Often fragments are expressed in the form of phage-coat fusion proteins. This manner of expression is advantageous for affinity-sharpening of antibodies.

### Humanized Antibodies

The invention further provides for humanized antibodies in which one or more of the CDRs are derived from a non-human antibody sequence, and one or more, but preferably all, of the CDRs bind specifically to the epitope of HSP70 set forth in SEQ 95%, or 99% sequence identity to this epitope.

A humanized antibody contains constant framework regions derived substantially from a human antibody (termed an acceptor antibody), as well as, in some instances, a majority of the variable region derived from a human antibody. One or more of the CDRs (all or a portion thereof, as well as discreet amino acids surrounding one or more of the CDRs) are provided from a non-human antibody, such as a mouse antibody. The constant region(s) of the antibody, may or may not be present. Humanized antibodies provide several advantages over non-humanized antibodies for therapeutic or diagnostic use in humans. These include:

1) The human immune system should not recognize the framework or constant region of the humanized antibody as foreign, and therefore the antibody response against such an injected antibody should be less than against a totally foreign mouse antibody or a partially foreign chimeric antibody;

2) Because the effector portion of the humanized antibody is human, it may interact better with other parts of the human immune system; and

3) Injected mouse antibodies have been reported to have a much shorter half-life in the human circulation than the half-life exhibited by normal human antibodies (see, e.g., Shaw et al., J Immunol 138:4534-4538 (1987)). Injected humanized antibodies have a half-life essentially equivalent to naturally occurring human antibodies, allowing smaller and less frequent doses.

The substitution of one or more mouse CDRs into a human variable domain framework is most likely to result in retention of their correct spatial orientation if the human variable domain framework adopts the same or similar conformation to the mouse variable framework from which the CDRs originated. This is achieved by obtaining the human variable domains from human antibodies whose framework sequences exhibit a high degree of sequence identity with the murine variable framework domains from which the CDRs were derived. The heavy and light chain variable framework regions can be derived from the same or different human antibody sequences. The human antibody sequences can be the sequences of naturally occurring human antibodies or can be consensus sequences of several human antibodies. See, e.g., Kettleborough et al., Protein Engineering 4:773 (1991); Kolbinger et al., Protein Engineering 6:971 (1993).

Suitable human antibody sequences are identified by computer comparisons of the amino acid sequences of the mouse variable regions with the sequences of known human antibodies. The comparison is performed separately for heavy and light chains but the principles are similar for each.

Methods of preparing chimeric and humanized antibodies and antibody fragments are described in U.S. Pat. Nos. 4,816,567, 5,530,101, 5,622,701, 5,800,815, 5,874,540, 5,914,110, 5,928,904, 6,210,670, 6,677,436, and 7,067,313 and U.S. Patent Application Nos. 2002/0031508, 2004/0265311, and 2005/0226876. Preparation of Antibody or Fragments Thereof is Further Described in U.S. Pat. Nos. 6,331,415, 6,818,216, and 7,067,313.

### Diagnostic or Therapeutic Agents of the Invention

Agents of the invention (e.g., peptide, polypeptides, antibodies, or small molecules) can be coupled to chelating compounds to form a diagnostic or therapeutic agent that can be used to detect, diagnose, or treat cancer cells according to the methods disclosed herein. Diagnostic and therapeutic agents may be prepared by various methods depending upon the chelator chosen. In addition, agents of the invention may be labeled with a fluorescent molecule to facilitate a diagnostic or therapeutic application of the invention.

Agents of the invention (e.g., peptides, polypeptides, or antibodies) may be coupled to form a conjugate by reacting the free amino group of the N-terminal residue of the agent with an appropriate functional group of the chelator, such as a carboxyl group or activated ester. For example, a conjugate may incorporate the chelator ethylenediaminetetraacetic acid (EDTA), common in the art of coordination chemistry, when functionalized with a carboxyl substituent on the ethylene chain. Synthesis of EDTA derivatives of this type are reported in Arya et al., (Bioconjugate Chemistry. 2:323, 1991), wherein the four coordinating carboxyl groups are each blocked with a t-butyl group while the carboxyl substituent on the ethylene chain is free to react with the amino group of the agent thereby forming a conjugate.

A conjugate may incorporate a metal chelator component that is peptidic, i.e., compatible with solid-phase peptide synthesis. In this case, the chelator may be coupled to the agent of the invention (e.g., peptide, polypeptide, antibody, or small molecule) in the same mariner as EDTA described above or more conveniently the chelator and agent are synthesized in tato starting from the C-terminal residue of the peptide and ending with the N-terminal residue of the chelator.

Conjugates may further incorporate a linker moiety that serves to couple the agent of the invention (e.g., peptide, polypeptide, antibody, or small molecule) to the chelator while not adversely affecting either the targeting function of the agent or the metal-binding function of the chelator. Suitable linking groups include amino acid chains and alkyl chains functionalized with reactive groups for coupling to both the agent and the chelator. An amino acid chain is the preferred linking group when the chelator is peptidic so that the conjugate can be synthesized in toto by solid-phase techniques.

An alkyl chain linking group may be incorporated in the conjugate by reacting the amino group of the N-terminal residue of the agent of the invention (e.g., peptide, polypeptide, or antibody) with a first functional group on the alkyl chain, such as a carboxyl group or an activated ester. Subsequently the chelator is attached to the alkyl chain to complete the formation of the conjugate by reacting a second functional group on the alkyl chain with an appropriate group on the chelator. The second functional group on the alkyl chain is selected from substituents that are reactive with a functional group on the chelator while not being reactive with the N-terminal residue of the agent. For example, when the chelator incorporates a functional group, such as a carboxyl group or an activated ester, the second functional group of the alkyl chain linking group can be an amino group. It will be appreciated that formation of the conjugate may require protection and deprotection of the functional groups present in order to avoid formation of undesired products. Protection and deprotection are accomplished using protecting groups, reagents, and protocols common in the art of organic synthesis. Particularly, protection and deprotection techniques employed in solid phase peptide synthesis described above may be used.

An alternative chemical linking group to an alkyl chain is polyethylene glycol (PEG), which is functionalized in the same manner as the alkyl chain described above for incorporation in the conjugates. The agents of the invention (e.g., peptides, polypeptides, antibodies, and small molecules) can be PEGylated for improved systemic half-life and reduced dosage frequency, it will be appreciated that linking groups may alternatively be coupled first to the chelator and then to the agent of the invention.

Another aspect of the invention involves cross-linking the agents of the invention (e.g., peptides, polypeptides, antibodies, or small molecules) to improve their pharmacokinetic, immunogenic, diagnostic, and/or therapeutic attributes. Cross-linking involves joining two molecules by a covalent bond through a chemical reaction at suitable site(s) (e.g., primary amines, sulfhydryls) on the agent of the invention. In an embodiment, one or more agents of the invention can be cross-linked together. Alternatively, cross-linked agents of the invention include but are not limited to hapten-carrier protein conjugates, antibody-enzyme conjugates, antibody-toxin conjugates (immunotoxins) and other labeled agents of the invention.

In accordance with another aspect of the invention, agent of the invention (e.g., peptide, polypeptide, antibody, or small molecule)-chelator conjugates may incorporate a diagnostically or therapeutically useful metal capable of forming a complex. Suitable metals include, e.g., radionuclides, such as technetium and rhenium in their various forms (e.g., ^{99 m}TcO³⁺, ^{99 m} TcO₂⁺, ReO³⁺, and ReO₂⁺). Incorporation of the metal within an agent-chelator conjugate can be achieved by various methods common in the art of coordination chemistry. When the metal is technetium-99 m, the following general procedure may be used to form a technetium complex. An agent-chelator conjugate solution is formed initially by dissolving the conjugate in aqueous alcohol such as ethanol. The solution is then degassed to remove oxygen then thiol protecting groups are removed with a suitable reagent, for example, with sodium hydroxide, and then neutralized with an organic acid, such as acetic acid (pH 6.0-6.5). In the labeling step, a stoichiometric excess of sodium pertechnetate, obtained from a molybdenum generator, is added to a solution of the conjugate with an amount of a reducing agent such as stannous chloride sufficient to reduce technetium and heated. The labeled conjugate may be separated from contaminants ^{99 m}TcO₄⁻ and colloidal ^{99 m}TcO₂ chromatographically, for example, with a C-18 Sep Pak cartridge.

In an alternative method, labeling can be accomplished by a transchelation reaction. The technetium source is a solution of technetium complexed with labile ligands facilitating ligand exchange with the selected chelator. Suitable ligands for transchelation include tartarate, citrate, and heptagluconate. In this instance the preferred reducing reagent is sodium dithionite. It will be appreciated that the conjugate may be labeled using the techniques described above, or alternatively the chelator itself may be labeled and subsequently coupled to the peptide, polypeptide, or antibody of the invention to form the conjugate; a process referred to as the “prelabeled ligand” method.

Another approach for labeling conjugates of the present invention involves immobilizing the agent-chelator conjugate on a solid-phase support through a linkage that is cleaved upon metal chelation. This is achieved when the' chelator is coupled to a functional group of the support by one of the complexing atoms. Preferably, a complexing sulfur atom is coupled to the support which is functionalized with a sulfur protecting group such as maleimide.

When labeled with a diagnostically or therapeutically useful metal, agent-chelator conjugates of the present invention can be used to detect neoplasms (e.g., lung cancer, breast cancer, colon cancer, and prostate cancer) by procedures established in the art of diagnostic imaging. A conjugate labeled with a radionuclide metal, such as technetium-99 m, may be administered to a mammal by intravenous injection in a pharmaceutically acceptable solution such as isotonic saline, or by other methods described herein. The amount of labeled conjugate appropriate for administration is dependent upon the distribution profile of the chosen conjugate in the sense that a rapidly cleared conjugate may be administered in higher doses than one that clears less rapidly. Unit doses acceptable for imaging neoplasms are in the range of about 5-40 mCi for a 70 kg individual. In vivo distribution and localization can be tracked by standard techniques described herein at an appropriate time subsequent to administration; typically between 30 minutes and 180 minutes depending upon the rate of accumulation at the target site with respect to the rate of clearance at non-target tissue.

The agents of the invention (e.g., peptides, polypeptides, antibodies, or small molecules) can be labeled for fluorescence detection by labeling the agent with a fluorophore, such as rhodamine or fluorescein, using techniques well known in the art (see, e.g., Lohse et al., Bioconj Chem 8:503-509 (1997)). Using techniques well known in the art, cancer targeting agents of the invention can also be labeled with a radioactive metal or a relaxivity metal by coupling the agent to a metal chelating agent that chelates a radioactive metal or relaxivity metal. Examples of chelating agents include, but are not limited to, ininocarboxylic and polyaminopolycarboxylic reactive groups, diethylenetriaminepentaacetic acid (DTPA), and 1,4,7,10-tetraazacyclododecane-1,4,7,10-tetraacetic acid (DOTA). The chelating agent can be coupled via its amino acid side chain directly to the cancer targeting agent. Alternatively, an intervening amino acid sequence or linker moiety can be use to couple the cancer targeting agent to the chelating agent.

### Therapeutic or Cytotoxic Agents of the Invention

An agent of the invention (e.g., peptide, polypeptide, antibody, or small molecule) can be prepared by coupling the agent to any known cytotoxic or therapeutic moiety. Examples of therapeutic or cytotoxic agents that can be coupled to an agent of the invention include, e.g., antineoplastic agents such as: Acivicin; Aclarubicin; Acodazole Hydrochloride; Acronine; Adozelesin; Adriamycin; Aldesleukin; Altretamine; Ambomycin; A. metantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Camptothecin; Caracemide; Carbetimer; Carboplatin; Carmustine; Carubicin Hydrochloride; Carzelesin; Cedefingol; Chlorambucil; Cirolemycin; Cisplatin; Cladribine; Combretestatin A-4; Crisnatol Mesylate; Cyclophosphamide; Cytarabine; Dacarbazine; DACA (N-[2-(Dimethyl-amino) ethyl] acridine-4-carboxamide); Dactinomycin; Daunorubicin Hydrochloride; Daunomycin; Decitabine; Dexormaplatin; Dezaguanine; Dezaguanine Mesylate; Diaziquone; Docetaxel; Dolasatins; Doxorubicin; Doxorubicin Hydrochloride; Droloxifene; Droloxifene Citrate; Dromostanolone Propionate; Duazomycin; Edatrexate; Eflornithine Hydrochloride; Ellipticine; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Fluorocitabine; Fosquidone; Fostriecin Sodium; Gemcitabine; Gemcitabine Hydrochloride; Gold Au 198; Homocamptothecin; Hydroxyurea; Idarubicin Hydrochloride; Ifosfamide; Ilmofosine; Interferon Alfa-2a; Interferon Alfa-2b; Interferon Alfa-nl; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; PeploycinSulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Rhizoxin; Rhizoxin D; Riboprine; Rogletimide; Safingol; Safingol Hydrochloride; Semustine; Simtrazene; Spartbsate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP53; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′ Deoxyformycin; 9-aminocamptothecin; raltitrexed; N-propargyl-5,8-dideazafolic acid; 2chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlor ethamine); cyclophosphamide; melphalan; chlorambucil; ifosfamide; busulfan; N-methyl-Nnitrosourea (MNU); N,N′-Bis (2-chloroethyl)-N-nitrosourea (BCNU); N-(2-chloroethyl)-N′ cyclohexyl-N-nitrosourea (CCNU); N-(2-chloroethyl)-N′-(trans-4-methylcyclohexyl-N-nitrosourea (MeCCNU); N-(2-chloroethyl)-N′-(diethyl)ethylphosphonate-N-nitrosourea (fotemustine); streptozotocin; diacarbazine (DTIC); mitozolomide; temozolomide; thiotepa; mitomycin C; AZQ; adozelesin; Cisplatin; Carboplatin; Ormaplatin; Oxaliplatin; Cl-973; DWA 2114R; JIv1216; JM335; Bis (platinum); tomudex; azacitidine; cytarabine; gemcitabine; 6-Mercaptopurine; 6-Thioguanine; Hypoxanthine; teniposide 9-amino camptothecin; Topotecan; CPT-11; Doxorubicin; Daunomycin; Epirubicin; darubicin; mitoxantrone; losoxantrone; Dactinomycin (Actinomycin D); amsacrine; pyrazoloacridine; all-trans retinol; 14-hydroxy-retro-retinol; all-trans retinoic acid; N-(4-Hydroxyphenyl) retinamide; 13-cis retinoic acid; 3-Methyl TTNEB; 9-cis retinoic acid; fludarabine (2-F-ara-AMP); or 2-chlorodeoxyadenosine (2-Cda).

Other anti-neoplastic compounds include, but are not limited to, 20-pi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; argininedeaminase; asulacrine; atamestane; atrimustine; axinastatin 1; axinastatin 2; axinastatin 3; azasetron; azatoxin; azatvrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bleomycin A2; bleomycin B2; bropirimine; budotitane; buthionine sulfoximine; calcipotriol; calphostin C; camptothecin derivatives (e.g., 10-hydroxy-camptothecin); canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; 2′deoxycoformycin (DCF); deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; discodermolide; docosanol; dolasetron; doxifluridine; droloxifene; dronabinol; duocarmycin SA; ebselen; ecomustine; edelfosine; edrecolomab; eflomithine; elemene; emitefur; epirubicin; epothilones (A, R═H; B, R=Me); epithilones; epristeride; estramustine analogue; estrogen agonists; estrogen antagonists; etanidazole; etoposide; etoposide 4′-phosphate (etopofos); exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; homoharringtonine (HHT); hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maytansine; mannostatin A; marimastat; masoprocol; maspin; matrilysin inhibitors; matrix metalloproteinase inhibitors; menogaril; merbarone; meterelin; methioninase; metoclopramide; MIF inhibitor; ifepristone; miltefosine; mirimostim; mismatched double stranded RNA; mithracin; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; podophyllotoxin; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RIIretinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Compositions of the invention can also be prepared by coupling an agent of the invention to an antiproliferative agent; for example piritrexim isothionate. Alternatively, agents of the invention can be coupled to an antiprostatic hypertrophy agent such as, for example; sitogluside, a benign prostatic hyperplasia therapy agent such as, for example, tamsulosin hydrochloride, or a prostate growth inhibitor such as, for example, pentomone.

An agent of the invention (e.g., peptide, polypeptide, antibody, or small molecule) can also be coupled to a radioactive agent, including, but not limited to: Fibrinogen ¹²⁵I; Fludeoxyglucose ¹⁸F; Fluorodopa ¹⁸F; Insulin ¹²⁵I; Insulin ¹³¹I; lobenguane ¹²³I; Iodipamide Sodium ¹³¹I; Iodoantipyrine ¹³¹I; Iodocholesterol ¹³¹I; Iodohippurate Sodium ¹²³I; Iodohippurate Sodium ¹²⁵I; Iodohippurate Sodium ¹³¹I; Iodopyracet ¹²⁵I; Iodopyracet ¹³¹I; lofetamine Hydrochloride ¹²³I; lomethin ¹²⁵I; lomethin ¹³¹I; lothalamate Sodium ¹²⁵I; lothalamate Sodium ¹³¹I; tyrosine ¹³¹I; Liothyronine ¹²⁵I; Liothyronine ¹³¹I; Merisoprol Acetate ¹⁹⁷Hg; Merisoprol Acetate ²⁰³Hg; Merisoprol ¹⁹⁷Hg; Selenomethionine ⁷⁵Se; Technetium ^{99m}Tc Antimony Trisulfide Colloid; Technetium ^{99m}Tc Bicisate; Technetium ^{99m}Tc Disofenin; Technetium ^{99m}Tc Etidronate; Technetium ^{99m}Tc Exametazime; Technetium ^{99m}Tc Furifosmin; Technetium ^{99m}Tc Gluceptate; Technetium ^{99m}Tc Lidofenin; Technetium ^{99m}Tc Mebrofenin; Technetium ^{99m}Tc Medronate; Technetium ^{99m}Tc Medronate Disodium; Technetium ^{99m}Tc Mertiatide; Technetium ^{99m}Tc Oxidronate; Technetium ^{99m}Tc Pentetate; Technetium ^{99m}Tc Pentetate Calcium Trisodium; Technetium ^{99m}Tc Sestamibi; Technetium ^{99m}Tc Siboroxime; Technetium ^{99m}Tc; Succimer; Technetium ^{99m}Tc Sulfur Colloid; Technetium ^{99m}Tc Teboroxime; Technetium ^{99m}Tc Tetrofosmin; Technetium ^{99m}Tc Tiatide; Thyroxine ¹²⁵I; Thyroxine ¹³¹I; Tolpovidone ¹³¹I; Triolein ¹²⁵I; or Triolein ¹³¹I.

Therapeutic or cytotoxic agents of the invention may further include a peptide, polypeptide, antibody, or small molecule of the invention coupled to, for example, an anti-cancer Supplementary Potentiating Agent, including, but not limited to: Tricyclic anti-depressant drugs (e.g., imipramine, desipramine, amitryptyline, clomipramine, trimipramine, doxepin, nortriptyline, protriptyline, amoxapine, and maprotiline); non-tricyclic anti-depressant drugs (e.g., sertraline, trazodone, and citalopram); Ca++ antagonists (e.g., verapamil, nifedipine, nitrendipine, and caroverine); Calmodulin inhibitors (e.g., prenylamine, trifluoroperazine, and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and Multiple Drug Resistance reducing agents such as Cremaphor EL.

An agent of the invention (e.g., peptide, polypeptide, antibody, or small molecule) can also be administered with cytokines such as granulocyte colony stimulating factor.

The agents of the invention (e.g., peptides, polypeptides, antibodies, and small molecules) may also be administered in anti-cancer cocktails that include (some with their MTDs shown in parentheses): gemcitabine (1000 mg/m²); methotrexate (15 gm/m² i.v.+leuco.<500 mg/m² i.v. w/o leuco); 5-FU (500 mg/m²/day×5 days); FUDR (100 mg/kg×5 in mice, 0.6 mg/kg/day in human i.a.); FdUMP; Hydroxyurea (35 mg/kg/d in man); Docetaxel (60-100 mg/m²); discodermolide; epothilones; vincristine (1.4 mg/m²); vinblastine (escalating: 3.3-11.1 mg/m², or rarely to 18.5 mg/m²); vinorelbine (30 mg/m²/wk); meta-pac; irinotecan (50-150 mg/m², 1×/wk depending on patient response); SN-38 (−100 times more potent than Irinotecan); 10-OH campto; topotecan (1.5 mg/m²/day in humans, 1×iv LDIO mice=75 mg/m²); etoposide (100 mg/m² in man); adriamycin; flavopiridol; Cis-Pt (100 mg/m² in man); carbo-Pt (360 mg/m² in man); bleomycin (20 mg/m2); mitomycin C (20 mg/m²); mithramycin (30 sug/kg); capecitabine (2.5 g/m² orally); cytarabine (100 mg/m²/day); 2-Cl-2′ deoxyadenosine; Fludarabine-P04 (25 mg/m²/day, ×5 days); mitoxantrone (12-14 mg/m²); mitozolomide (>400 mg/m²); Pentostatin; or Tomudex.

An agent of the invention (e.g., peptide, polypeptide, antibody, or small molecule) can also be coupled to an antimetabolic agent, such as methotrexate. Antimetabolites include, but are not limited to, the following compounds and their derivatives: azathioprine, cladribine, cytarabine, dacarbazine, fludarabine phosphate, fluorouracil, gencitabine chlorhydrate, mercaptopurine; methotrexate, mitobronitol, mitotane, proguanil chlorohydrate, pyrimethamine, raltitrexed, trimetrexate glucuronate, urethane, vinblastine sulfate, vincristine sulfate, etc. More preferably, X may be a folic acid-type antimetabolite, a class of agents that includes, for example, methotrexate, proguanil chlorhydrate, pyrimethanime, trimethoprime, or trimetrexate glucuronate, or derivatives of these compounds.

In another embodiment, the agent of the invention (e.g., peptide, polypeptide, antibody, or small molecule) may be coupled to a member of the anthracycline family of neoplastic agents, including but not limited to aclarubicine chlorhydrate, daunorubicine chlorhydrate, doxorubicine chlorhydrate, epirubicine chlorhydrate, idarubicine chlorhydrate, pirarubicine, or zorubicine chlorhydrate; a camptothecin, or its derivatives or related compounds, such as 10, 11 methylenedioxycamptothecin; or a member of the maytansinoid family of compounds, which includes a variety of structurally related compounds, e.g., ansamitocin P3, maytansine, 2′-N-demethylmaytanbutine, and maytanbicyclinol.

The agent of the invention (e.g., peptide, polypeptide, antibody, or small molecule) can be modified or labeled to facilitate diagnostic or therapeutic uses. Detectable labels such as a radioactive, fluorescent, heavy metal, or other agents may be bound to the peptide, polypeptide, antibody, or small molecule of the invention. Single, dual, or multiple labeling of an agent may be advantageous. For example, dual to labeling with radioactive iodination of one or more residues combined with the additional coupling of, for example, ⁹⁰Y via a chelating group to amine-containing side or reactive groups, would allow combination labeling. This may be useful for specialized diagnostic needs such as identification of widely dispersed small neoplastic cell masses.

An agent of the invention (e.g., peptide, polypeptide, antibody, or small molecule), or an analog thereof, may also be modified, for example, by halogenation of the tyrosine residues of the peptide component. Halogens include fluorine, chlorine, bromine, iodine, and astatine. Such halogenated agents of the invention may be detectably labeled, e.g., if the halogen is a radioisotope, such as, for example, ¹⁸F, ⁷⁵Br, ⁷⁷Br, ¹²²I, ¹²³I, ¹²⁴I, ¹²⁵I, ¹²⁹I, ¹³¹I, or ²¹¹At. Halogenated agents of the invention contain a halogen covalently bound to at least one amino acid, and preferably to D-Tyr residues present in the agent. Other suitable detectable modifications include binding of other compounds (e.g., a fluorochrome such as fluorescein) to a lysine residue of the analog, particularly an analog having a linker including lysines.

Radioisotopes for radiolabeling an agent of the invention (e.g., peptide, polypeptide, antibody, or small molecule) of the invention include any radioisotope that can be covalently bound to an amino acid residue of the agent of the invention, or an analog thereof, or a suitable reactive group. The radioisotopes can be selected from radioisotopes that emit either beta or gamma radiation, or alternatively, the agent can be modified to contain a chelating group that, for example, can be covalently bonded to a lysine residue(s) within the agent. The chelating group can then be modified to contain any of a variety of radioisotopes, such as gallium, indium, technetium, ytterbium, rhenium, or thallium (e.g., ¹²⁵I, ⁶⁷Ga, ¹¹¹In, ⁹⁹mTc, ¹⁶⁹Yb, ¹⁸⁶Re).

Where the agent of the invention is modified by attachment of a radioisotope, preferably the radioisotope is one having a radioactive half-life corresponding to, or longer than, the biological half-life of the agent used. More preferably, the radioisotope is a radioisotope of a halogen atom (e.g. a radioisotope of fluorine, chlorine, bromine, iodine, and astatine), even more preferably ⁷⁵Br, ⁷⁷Br, ⁷⁶Br, ¹²²I, ¹²³I, ¹²⁴I, ¹²⁵I; ¹²⁹I, ¹³¹I, or ²¹¹At.

Agents of the invention (e.g., peptides, polypeptides, antibodies, or small molecules) that include radioactive metals are useful in radiographic imaging or radiotherapy. Preferred radioisotopes also include ^{99m}Tc, ¹⁵Cr, ⁶⁷Ga, ⁶⁸Ga, ¹¹¹In, ¹⁶⁸I, ¹⁴⁰La, ⁹⁰Y, ⁸⁸Y, ¹⁵³Sm, ¹⁵⁶Ho, ¹⁶⁵Dy, ⁶⁴Cu, ⁹⁷Ru, ¹⁰³Ru, ¹⁸⁶Re, ¹⁸⁸Re, ²⁰³Pb, ²¹¹Bi, ²¹²Bi, ²¹³Bi, and ²¹⁴Bi. The choice of metal is determined based on the desired therapeutic or diagnostic application.

Agents of the invention (e.g., peptides, polypeptides, antibodies, or small molecules) that include a metal component are useful as diagnostic and/or therapeutic agents. A detectable label may be a metal ion from heavy elements or rare earth ions, such as Gd³⁺, Mn³⁺, or Cr²⁺. Conjugates that include paramagnetic or superparamagnetic metals are useful as diagnostic agents in MRI imaging applications. Paramagnetic metals that may be used in the peptide agents include, but are not limited to, chromium (III), manganese (II), iron (II), iron (III), cobalt (II), nickel (II), copper (II), praseodymium (III), neodymium (III), samarium (III), gadolinium (III), terbium (III), dysprosium (III), holmium (III), erbium (III), and ytterbium (III). Preferably, the metal has a relaxivity of at least 10, 12, 15, or 20 mM⁻¹ sec⁻¹ Z⁻¹, wherein Z is the concentration of paramagnetic metal. Chelating groups may be used to indirectly couple detectable labels or other molecules to the agents of the invention. Chelating groups may link the agents of the invention with radiolabels, using, e.g., a bifunctional stable chelator, or they may be linked to one or more terminal or internal amino acid reactive groups within the agent of the invention. They may be also linked via an isothiocyanate β-Ala or appropriate non α-amino acid linker which prevents Edman degradation. Examples of chelators known in the art include, for example, the ininocarboxylic and polyaminopolycarboxylic reactive groups, ininocarboxylic and polyaminopolycarboxylic reactive groups, diethylenetriaminepentaacetic acid (DTPA), and 1,4,7,10-tetraazacyclododecane-1,4,7,10-tetraacetic acid (DOTA). For general methods, see, e.g., Liu et al., Bioconjugate Chem. 12(4):653, 2001; Cheng et al., WO 89/12631; Kieffer et al., WO 93/12112; Albert et al., U.S. Pat. No. 5,753,627; and WO 91/01144 (each of which are hereby incorporated by reference).

### Pharmaceutical Compositions of the Invention

Agents of the invention (e.g., peptides, polypeptides, antibodies, or small molecules) can be formulated for administration to a patient for therapeutic or diagnostic use, or for diagnostic use in vitro. When coupled to a therapeutic or cytotoxic agent, the specific targeting by the agent of the invention allows selective destruction of tumors. For example, the agent of the invention can be used to target and destroy neoplasms of the lung, breast, prostate, and colon. An agent of the invention may be administered to a mammalian subject, such as a human, directly or in combination with any pharmaceutically acceptable carrier or salt known in the art. Pharmaceutically acceptable salts may include non-toxic acid addition salts or metal complexes that are commonly used in the pharmaceutical industry. Examples of acid addition salts include organic acids such as acetic, lactic, pamoic, maleic, citric, malic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acids such as hydrochloric acid, hydrobromic acid, sulfuric acid phosphoric acid, or the like. Metal complexes include zinc, iron, and the like. One exemplary pharmaceutically acceptable carrier is physiological saline. Other physiologically acceptable carriers and their formulations are known to one skilled in the art and described, for example, in Remington's Pharmaceutical Sciences. (18^{th} edition), ed. A. Gennaro, 1990, Mack Publishing Company, Easton, Pa.

Pharmaceutical formulations of a therapeutically effective amount of an agent of the invention (e.g., peptide, polypeptide, antibody, or small molecule), or pharmaceutically acceptable salt-thereof, can be administered orally, parenterally (e.g., intramuscular, intraperitoneal, intravenous, or subcutaneous injection, inhalation, intradermally, optical drops, or implant), nasally, vaginally, rectally, sublingually, or topically, in admixture with a pharmaceutically acceptable carrier adapted for the route of administration.

Methods well known in the art for making formulations are found, for example, in Remington's Pharmaceutical Sciences (18^{th} edition), ed. A. Gennaro, 1990, Mack Publishing Company, Easton, Pa. Compositions containing an agent of the invention, which is intended for oral use, may be prepared in solid or liquid forms according to any method known to the art for the manufacture of pharmaceutical compositions. Compositions containing an agent of the invention may optionally contain sweetening, flavoring, coloring, perfuming, and/or preserving agents in order to provide a more palatable preparation. Solid dosage forms for oral administration include capsules, tablets, pills, powders, and granules. In such solid forms, the agent may be admixed with at least one inert pharmaceutically acceptable carrier or excipient. These may include, for example, inert diluents, such as calcium carbonate, sodium carbonate, lactose, sucrose, starch, calcium phosphate, sodium phosphate, or kaolin. Binding agents, buffering agents, and/or lubricating agents (e.g., magnesium stearate) may also be used. Tablets and pills can additionally be prepared with enteric coatings.

Liquid dosage forms for oral administration of compositions containing an agent of the invention include pharmaceutically acceptable emulsions, solutions, suspensions, syrups, and soft gelatin capsules. These forms contain inert diluents commonly used in the art, such as water or an oil medium. Besides such inert diluents, compositions containing an agent of the invention can also include adjuvants, such as wetting agents, emulsifying agents, and suspending agents.

Formulations for parenteral administration of compositions containing an agent of the invention include sterile aqueous or non-aqueous solutions, suspensions, or emulsions. Examples of suitable vehicles include propylene glycol, polyethylene glycol, vegetable oils, gelatin, hydrogenated naphalenes, and injectable organic esters, such as ethyl oleate. Such formulations may also contain adjuvants, such as preserving, wetting, emulsifying, and dispersing agents. Biocompatible, biodegradable lactide polymer, lactide/glycolide copolymer, or polyoxyethylene-polyoxypropylene copolymers may be used to control the release of the compounds. Other potentially useful parenteral delivery systems for compositions containing an agent of the invention include ethylene-vinyl acetate copolymer particles, osmotic pumps, implantable infusion systems, and liposomes.

Liquid formulations can be sterilized by, for example, filtration through a bacteria-retaining filter, by incorporating sterilizing agents into the compositions, or by irradiating or heating the compositions. Alternatively, they can also be manufactured in the form of sterile, solid compositions which can be dissolved in sterile water or some other sterile injectable medium immediately before use.

Compositions containing an agent of the invention for rectal or vaginal administration are preferably suppositories which may contain, in addition to active substances, excipients such as coca butter or a suppository wax. Compositions for nasal or sublingual administration are also prepared with standard excipients known in the art. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops or spray, or as a gel.

The amount of active ingredient in the compositions of the invention can be varied. One skilled in the art will appreciate that the exact individual dosages may be adjusted somewhat depending upon a variety of factors, including the peptide being administered, the time of administration, the route of administration, the nature of the formulation; the rate of excretion, the nature of the subject's conditions, and the age, weight, health, and gender of the patient. In addition, the severity of the condition targeted by the agent will also have an impact on the dosage level. Generally, dosage levels of between 0.1 μg/kg to 100 mg/kg of body weight are administered daily as a single dose or divided into multiple doses. Preferably, the general dosage range is between 250 μg/kg to 5.0 mg/kg of body weight per day. Wide variations in the needed dosage are to be expected in view of the differing efficiencies of the various routes of administration. For instance, oral administration generally would be expected to require higher dosage levels than administration by intravenous injection. Variations in these dosage levels can be adjusted using standard empirical routines for optimization, which are well known in the art. In general, the precise therapeutically effective dosage will be determined by the attending physician in consideration of the above identified factors.

The agents of the invention (e.g., peptides, polypeptides, antibodies, and small molecules) can be administered in a sustained release composition, such as those described in, for example, U.S. Pat. No. 5,672,659 and U.S. Pat. No. 5,595,760. The use of immediate or sustained release compositions depends on the type of condition being treated. If the condition consists of an acute or over-acute disorder, a treatment with an immediate release form will be preferred over a prolonged release composition. Alternatively, for preventative or long-term treatments, a sustained released composition will generally be preferred.

The present invention is illustrated by the following examples, which are in no way intended to be limiting of the invention.

### EXAMPLES

### Example 1

### Therapeutic Effect of a Tumor Binding Peptide Labeled with a Beta Emitter

Yttrium labeled peptides will be prepared for the purpose of testing their potential therapeutic value in rats bearing xenograft tumors. Peptide will be joined to a chelator group, such as DOTA, via a short linker sequence (gly-gly-gly-ser) at the carboxy terminus. 90-YCl3 will be obtained from a commercial vendor. 90-Y will be reacted in ammonium acetate buffer at pH ˜4.5 with a solution of peptide-DOTA to label the peptide complex. Tumor bearing rats will be prepared by injecting 1.0 ml of 2 mg/ml cell suspension of lung cancer cells subcutaneously into each flank. Tumors will grow for approximately two weeks. Tumor bearing animals will be randomized to experimental and control groups. Peptide will be administered by tail vein injection to each animal. Five animals will receive a single dose of 5 mCi/kg of 90-Y-peptide, five animals will receive a single dose of 10 mCi/kg of 90-Y-peptide, and five control animals will receive a single equimolar dose of unlabeled peptide (8 nmol/kg). Volumes of individual tumors will be calculated using a caliper to determine the three largest diameters, d1, d2, and d3, according to the formula for an ellipsoid, V=(π/6) (d1*d2*d3). The tumor volume for each animal will be determined as the sum of the volumes of the individual tumors on each animal. Tumor volumes will be determined every three to four days. Tumor volumes, normalized to the initial tumor volumes, will be plotted as a function of time.

The control animal group will be sacrificed once tumors become 2 cc in volume or necrotic; this term is expected to be approximately ten days post peptide injection. Out of the group receiving 5 mCi/kg, one animal is expected to show complete remission by three weeks post injection while the other four will show a delay in growth of approximately one week in comparison to controls. Out of the group of animals receiving 10 mCi/kg, four are expected to show complete remission by seven weeks post injection without regrowth for an observation period of ˜8 months. One animal will show a delay in growth, but will require sacrifice once the tumor reaches a volume of 2 cc or becomes necrotic.

### Example 2

### Human Study to Test the Diagnostic Use of a Tumor Binding Peptide Labeled with a Gamma Emitter

Fifteen patients with diagnosed stage 3b or stage 4 lung cancers will be enrolled. Patients less than 18 years of age or pregnant patients will be excluded. The anticipated mean age of study subjects is 60 years. Each study subject will undergo PET/CT imaging according to standard clinical protocol which typically involves administration of 10-13 mCi of 18-FDG followed by PET and non-contrast CT imaging from the base of the skull to the proximal thighs. Between one and two weeks following PET imaging, scintigraphic images using radio-labeled peptide as the radiopharmaceutical will be obtained. Radiopharmaceutical will be prepared by reacting peptide-gly-gly-gly-ser-DTPA with 111-In chloride. Each study subject will receive an intravenously administered dose of 5 mCi of 111-In labeled peptide. Anterior and posterior whole body planar images of the subjects will be obtained at 24 and 48 hours following administration of the radio labeled peptide. SPECT imaging of the chest and abdomen will also be obtained at 24 and 48 hours following administration of the radio labeled peptide. Scintigraphic images will be acquired on a SPECT/CT camera using a medium energy collimator. Two radiologists will read the PET/CT study of each subject and two other radiologists will read the SPECT/CT study of each subject; each set of radiologists will be blinded to the results of the other study. Location, size, and extent of each abnormal focus of radiotracer uptake by PET imaging will be compared with the same by SPECT imaging. Cohen's kappa testing will be used to ascertain the level of agreement between PET and SPECT imaging.

Among the 15 study subjects, we expect to observe forty abnormal foci of radiotracer uptake by both PET and SPECT imaging. Approximately 9 patients will be stage 3b by each imaging modality. By comparing SPECT images obtained at 48 hours with PET images, a Cohen's kappa ratio of 0.85 will be calculated. The Cohen's kappa ratio obtained by comparing 24 hour SPECT images with PET will be 0.8. Agreement will not vary significantly by anatomic region, organ of location, or size of the lesion. In comparing overall staging of cancer, a Cohen's kappa ratio of 0.95 will be calculated for the two imaging modalities.

### Example 3

### Identification of a Cancer Binding Peptide and Epitope

### Selection of Cancer Binding Peptides

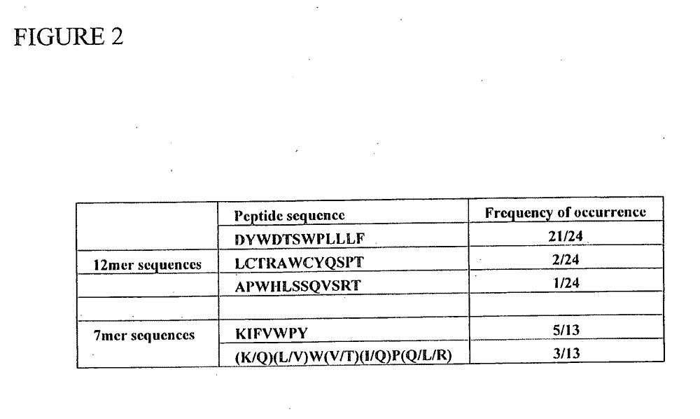

Following the final round of panning against cancer cells, 24 clones from the unamplified 12mer phage pool and 13 clones from the unamplified 7mer phage pool were sequenced. FIG. 2 shows the sequences that were obtained with the highest degree of consensus.

### Cell Internalization of Cancer Binding Peptide

Cancer cell binding 12mer was labeled with fluorescein (PanF) and used to incubate lung cancer (NCI-H460), prostate cancer (DU-145), and fibroblasts (CCD-1070Sk) grown in culture. Fluorescent microscopic visualization revealed significant cellular uptake by lung cancer and prostate cancer cells, but significant uptake wasn't seen in the case of fibroblasts.

### Cytotoxicity of Cancer Binding Peptide Conjugated to a Lytic Peptide Sequence

Cancer binding 12mer conjugated to a lytic peptide sequence via a 4 residue linker (PanL) was synthesized and tested for differential cytotoxic activity in lung cancer, prostate cancer, nonmalignant prostate epithelial cells, and fibroblasts. A dose dependent increase in cell death was observed in the case of each type of cancer cell up to a peptide concentration of 2.5 _{E} μM. A smaller degree of cell death was seen in the nonmalignant cell lines.

### In Vitro Cytotoxicity: Conjugation to Ricin-A

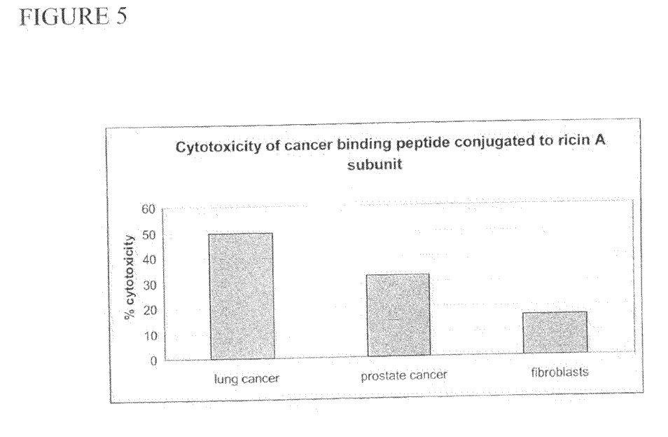

The percentage of each cell type killed is shown in FIG. 5. More cell killing was observed in the case of lung cancer (50%) and prostate cancer (32%) than fibroblasts (16%). Unconjugated ricin A subunit was also incubated with each cell type, and no cell killing was observed.

### Identification of Peptide Targets

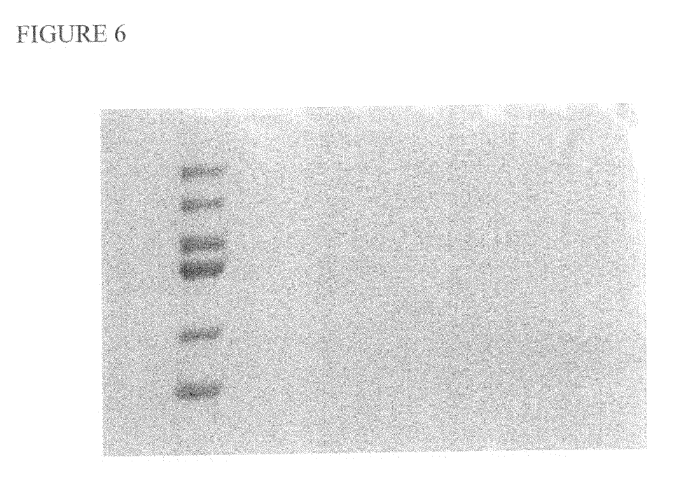

The cancer binding peptide conjugated to biotin (DYWDTSWPLLLFGGGSK; SEQ ID NO:12; PanB) was used in an affinity capture technique to isolate and identify the molecular targets to which the cancer-selecting peptide binds. The to species captured by affinity chromatography were eluted, and the eluate was further purified using SDS PAGE. A single, prominent band corresponding to 70kD (FIG. 6) appeared upon Coomassie blue staining. The band was excised and submitted for mass spectrometric analysis which revealed multiple related species. Among the identified targets were several members of the HSP70 family of heat shock proteins, including HSPA5; Stress-70 protein, mitochondrial precursor; isoform 1 of Heat shock cognate 71 kDa protein, and Heat shock 70 kDa protein 1. In addition protein disulfide isomerase A4 precursor was identified. Many of these proteins have been implicated in the unfolded protein response known to occur in cancer cells.

### Binding of Peptide to Selected Epitope of Hsc71

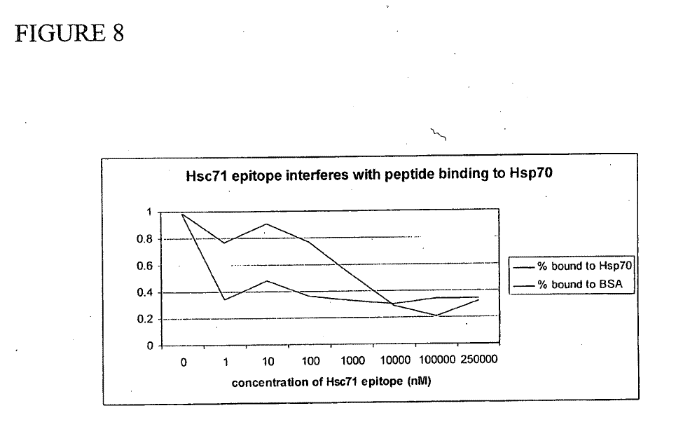

Higher levels of radio labeled peptide PanC were observed to bind to biotin-conjugated epitope (GIPPAPRGVPQIEVTF; SEQ ID NO:15) as the ratio of radio labeled to unlabeled PanC was increased (FIG. 8). The concentration of labeled PanC was held constant at 20 nM, while increasing amounts of unlabeled PanC were added to the mixture to block the binding of labeled peptide. Saturation of binding of hot PanC to the candidate epitope occurred above a labeled/unlabeled ratio of 0.1%.

Epitope of Hsc71 Interferes with Binding of Peptide to Hsp70

A synthetic epitope of Hsc71 (GIPPAPRGVPQIEVTF; SEQ ID 15) at various concentrations was added with a constant amount of PanC to Hsp70 coated wells to determine whether the epitope competes with Hsp70 for binding to PanC. The epitope suppressed binding of PanC to Hsp70 in a concentration dependent manner, consistent with specific binding competition between the epitope and Hsp70 for PanC binding (FIG. 8). By comparison, the epitope suppressed binding of PanC to albumin approximately equally at all concentrations, suggesting an absence of specific binding competition between the epitope and albumin. Taken together, these results suggest that PanC binding to Hsp70 is specifically mediated by binding to the selected epitope.

### Materials and Methods

Cell Culture

All cells were obtained from American Type Culture Collection (Manassas, Va.). Each cell culture was grown at 37C in 5% CO2. COLO 320DM (colon cancer) and NCl-1-1460 (lung cancer) were grown in RPMI 1640 medium with 2 mM L-glutamine adjusted to contain 1.5 g/L sodium bicarbonate, 4.5 g/L glucose, 10 mM HEPES, and 1.0 mM sodium pyruvate, and supplemented with 10% fetal bovine serum. DU-145 (prostate cancer) and CCD-1070Sk (fibroblasts) were grown in minimum essential medium (Eagle) with 2 mM L-glutamine and Earle's BSS adjusted to contain 1.5 g/L sodium bicarbonate, 0.1 mM non-essential amino acids, and 1.0 mM sodium pyruvate, and supplemented with 10% fetal bovine serum. Hs 578T (breast cancer) was grown in Dulbecco's modified Eagle's medium with 4 mM L-glutamine adjusted to contain 1.5 g/L sodium bicarbonate and 4.5 g/L glucose and supplemented with 0.01 mg/ml bovine insulin and 10% fetal bovine serum. RWPE-1 (non cancerous prostate epithelial cells) were grown in keratinocyte-serum free medium supplemented with 5 ng/ml human recombinant EGF and 0.05 mg/mL bovine pituitary extract.

Selection of Cancer Cell Binding Peptides

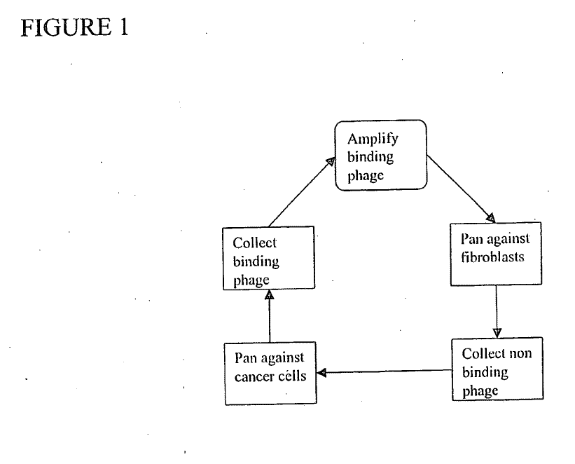

Phage display libraries (Ph.D.-12 and Ph.D.-7, New England Biolabs, Ipswich, Mass.) were serially panned against multiple cancer cell types. After panning against each cancer cell type, cancer cell binding clones were collected and amplified; the amplified clones were used for panning against normal fibroblasts. Following panning against normal fibroblasts, nonbinding clones were collected and used for panning against the next cancer cell type (see FIG. 1). In this way, clones were selected for their ability to bind multiple cancer cell types without binding normal fibroblasts. For the 12mer library, the order of cell types used for panning was 1) colon cancer to followed by fibroblasts, 2) prostate cancer followed by fibroblasts, 3) lung cancer followed by fibroblasts, 4) breast cancer followed by fibroblasts, 5) colon cancer followed by fibroblasts, and 6) prostate cancer followed by fibroblasts. The order of panning for the 7mer library was 1) breast cancer followed by fibroblasts, 2) lung cancer followed by fibroblasts, 3) prostate cancer followed by fibroblasts, and 4) colon cancer. A more detailed description of the methods is given below.

Tumor cells were trypsinized and suspended in PBS, pH 7.4. Cell suspensions were diluted to 10⁶ cells per mL. An aliquot (10 μL) of phage library was added to the cell suspension in a 1.5 mL Eppendorf tube and incubated for 60 minutes at 23C with shaking. Following incubation, cell suspensions were centrifuged at 18K rpm for 10 minutes. If the cell pellet was derived from cancer cells, the supernatant was discarded; the pellet was washed 3× with PBS and incubated in cell lysis solution (tris 25 mM, pH 7.5, 0.5% triton X-100). The lysis mixture was added to 20 mL of E. coli (ER2738) culture in LB medium during early log phase, and the Ph.D. library kit (New England Biolabs) manufacturer's instructions were followed for phage amplification. If the pellet was derived from normal fibroblasts, the supernatant (which contained nonbinding phage) was retained and used for subsequent panning against the next cancer cell culture in the series.

Following the final round of panning against cancer cells, the cells were pelleted, washed three times with PBS, and lysed. The phage containing lysis mixture was titered on agar plates according to the manufacturer's instructions. In case of the Ph.D.-12 library, 24 plaques were selected for DNA sequencing. In case of the Ph.D.-7 library, 15 plaques were selected. Consensus binding sequences are shown in FIG. 2.

Peptide Synthesis

Peptides were synthesized using standard FMOC protected chemistry. For in vitro studies, a cancer cell binding, 12 residue peptide sequence followed by a C-terminal, four residue linker sequence was labeled with fluorophores or cytotoxic agents. The sequence DYWDTSWPLLLFGGGSK (SEQ ID NO:12; PanF) (Flourescein)-amide was used for in vitro cell internalization studies. For in vitro cell killing studies, peptide was synthesized with a C-terminal, cell lytic sequence according to the following: DYWDTSWPLLLFGGGS(KFAKFAK)₃ (PanL; SEQ ID NO:13). The peptide sequence DYWDTSWPLLLFGGGC (PanC; SEQ ID NO:14) was synthesized for subsequent conjugation to ricin-A subunit and for radiolabeling with 99m-technetium. The peptide DYWDTSWPLLLFGGGSK-biotin (SEQ ID NO:12; PanB) was synthesized for cancer antigen isolation.

Cell Internalization

NCI-H640, DU-145, and CCD-1070Sk were grown in culture as detailed above. Cells were grown on coverslips in 6 well plates. A 25 μM solution of peptide DYWDTSWPLLLGGGSK-fluorescein (PanF; SEQ ID NO:12) was prepared in modified Eagle's medium. Cell culture media were replaced with 1.6 mL of peptide-containing medium, and the cells were incubated with peptide for one hour at 37C. Medium was removed and cells were washed three times with PBS. Two mL of 2% formaldehyde in PBS was added to each well, and plates were kept on ice for 15 minutes. Cells were washed with cold PBS three times and rinsed with distilled water. Coverslips were mounted with DAPI containing media and viewed with an Olympus BX51 fluorescent microscope and DP70 digital camera with excitation and emission wavelengths of 490 and 520 nm.

Cytotoxicity of Cancer Binding Peptide Conjugated to a Lytic Peptide Sequence

NCI-H640, DU-145, and CCD-1070Sk were grown in culture as detailed above. Cells were trypsinized and suspended in culture media containing 10% FBS. Cells were centrifuged at 1,000 rpm for 5 min. Supernatant was removed and cells were resuspended in 1 ml media without FBS. Cell suspensions were diluted to 20,000 cells/754. Twenty-five microliters of appropriately diluted peptide solution (PanL) was added to cell samples to give various drug concentrations of 0, 0.1, 0.5, 1.0, 2.5, 5.0 or 10 μM. Each sample was prepared in triplicate. Cell suspensions were transferred to a 96 well plate and incubated in the presence of various concentrations of PanL for 2 hours at 37C. Six samples of each cell type contained no peptide. The assay plate was removed from the incubator, and 24 of lysis solution (Tris 25 mM, pH 7.5, 0.5% triton X-100) was added to three samples of each cell type without peptide to generate a positive control maximum LDH release. LDH release was measured in each sample by adding 100 μl of CytoTox-ONE Reagent (Roche Applied Science) to each well and mixing on a plate shaker for 30 seconds. Samples were incubated at 22° C. for 10 minutes. The reaction was terminated by adding 50 μl of Stop Solution (per 100 μl of CytoTox-ONE™ Reagent added) to each well. Fluorescence was measured in each well using an excitation wavelength of 530 nm and an emission wavelength of 620 nm (Cytofluor 4000). The CytoTox-ONE assay was shown to yield a quantity of fluorescent product that is linearly proportional to the number of cells killed (correlation coefficient=0.99, data not shown). The percentage of cells killed was calculated using the following formula:

$$\% cytotoxicity = \frac{100 \% \times ( P - C )}{( M - C )}$$

where P=LDH release in wells of peptide incubated cells; C=LDH release in wells of cells not incubated with peptide; and M=LDH release in wells incubated in lysis solution. The formula is based upon the assumptions that, in a linear relationship between CytoTox-ONE product development and number of cells killed, C is the y-intercept, and M is due to 100% cell killing.

In Vitro Cytotoxicity: Conjugation to Ricin-A

Peptide PanC (DYWDTSWPLLLFGGGC; SEQ ID NO:14) was conjugated to ricin A subunit (Sigma-Aldrich). Ricin A was obtained from manufacturer in solution. A buffer exchange was performed with 0.1M PBS/20% glycerol. Ricin A was conjugated with NHS-PEO₄-maleimide cross linker (Pierce, Rockford, Ill.) at a 1:10 molar ratio for 30 minutes at room temperature. Derivatized ricin A was purified on a P4 column using 0.1M PBS/20% glycerol as an elution buffer. Derivatized ricin A was combined with P 12S at a 1:1 molar ratio and reacted for 2 hours at room temperature.

NCI-H640, DU-145, and CCD-1070Sk were grown in culture as detailed above. Cells were trypsinized and suspended in culture media containing 10% FBS. Cells were centrifuged at 1,000 rpm for 5 min. Supernatant was removed and cells were resuspended in 1 ml media without FBS. Cell suspensions were diluted to 20,000 cells/75 μl. Twenty-five microliters of appropriately diluted peptide-ricin A conjugate was added to each cell sample to give a drug concentration of 1 μM. Each sample was prepared in triplicate. Cell suspensions were transferred to a 96 well plate and incubated in the presence of peptide-ricin A conjugate for 2 hours at 37C. Percentage of cells killed (FIG. 5) was determined as outlined above.

Identification of Cancer Cell Antigen

NCI-H460 cells were cultured as above to generate 10⁹ cells. The CNM Protein Extraction Kit (BioChain, Hayward, Calif.) was used to extract cell membrane proteins. The manufacturer's instructions were modified to retain mitochondria in the cytosolic fraction; otherwise, extraction was performed according to the manufacturer's directions. The cell membrane protein-containing fraction was pooled and stored at −80C. Immobilized Neutravidin Columns were prepared by suspending immobilized Neutravidin beads in TBS pH 7.2 and pipeting 2004 of the resulting gel slurry into Nanosep 3K spin columns (Pall Corporation, East Hills, N.Y.). The columns were placed in collection tubes and 500 μl of TBS was added to each of the spin columns before centrifuging at 1,250×g for 30-60 seconds with the column cap open. Bottom plugs were then applied to each column.

Biotinylated peptide (PanB) was prepared as a 100 μg/mL solution; 5004 of solution was added to the spin columns. Columns were incubated at room temperature for 30 minutes with gentle rocking. Columns in collecting tubes were centrifuged at 1,250×g for 60 seconds to remove unbound PanB. Spin columns were to then transferred to separate collection tubes for biotin blocking. Two hundred fifty μL, of biotin blocking solution (100 μg/mL) was added to each spin column. Columns were capped and inverted 3-5 times followed by incubation at room temperature for 5 minutes. Top screw caps of each column were removed and columns were placed in collection tubes for centrifugation at 1,250×g for 30-60 seconds. TBS (pH 2.2) 500 μL was added to each spin cup. Top screw caps were replaced on each column and columns were inverted 3-5 times. Top screw caps were removed. Columns were placed in collection tubes and centrifuged at 1,250×g for 30-60 seconds. Columns were washed two additional times with 5004 of TBS before applying bottom plugs.

Cell membrane protein mixture was thawed to room temperature, and added to spin columns followed by incubation at 4° C. for 4 hrs with gentle rocking. Following incubation, top caps and bottom plugs were removed from each column. Columns were placed in collection tubes and centrifuged at 1,250×g for 60 seconds. Columns were transferred to separate collection tubes for washing. Columns were washed four times in TBS. After each wash, washing buffer was collected as waste in collection tubes following column centrifugation at 1,250×g for 30-60 seconds. Spin columns were transferred to fresh collection tubes for elution of captured membrane protein. Protein was eluted by incubating columns in elution buffer (0.2 M glycine-HCl, pH 2.2) for 3-5 minutes at room temperature followed by centrifugation at 1,250×g for 30-60 seconds. The eluate pH was neutralized and a buffer exchange was performed resulting in protein eluate in tris buffered saline, pH 7.2. The protein eluate was further purified using ID SDS polyacrylamide gel electrophoresis (PAGE) followed by Coomassie blue staining. A prominent band that appeared at the 70 kD position (FIG. 6) was excised and submitted to a protein sequencing core at University of Massachusetts Medical School. Protein was identified using mass spectrometry.

Radio Labeling of Peptide PanC

A 24 aliquot of PanC (3M) was mixed with 40 μl of 0.25M ammonium acetate, 15 μL of tartrate buffer pH 8.7, 4 μL of stannous chloride in 100 mM of sodium tartrate, and 30 μL of 99m-Tc pertechnetate. The mixture was heated for 25 minutes at 95C. QC was done with Sep-Pak and was always above 90%. A small aliquot was also injected on a Waters 600 HPLC to check the radiologic profile. Fractions were collected and read on a gamma counter (Perkin-Elmer Wallac Wizard 1470).

Binding of Peptide to Selected Epitope of Hsc71

In order to predict a specific epitope of the cancer cell antigens to which synthetic peptide PanC is binding, the amino acid sequences of the four HSP70 family members (identified above by antigen capture) were aligned using commercial software, and conserved regions were identified. The longest region which is conserved among the four proteins occurs at amino acids 463-478 of Hsc71 (GIPPAPRGVPQIEVTF; SEQ ID NO:15). This region is 78% longer than the next longest conserved region that was identified and so was determined to be a reasonable candidate epitope for the binding of PanC. This candidate epitope was synthesized using standard FMOC chemistry with biotin conjugated to the N terminus.

In order to test the binding of radio labeled PanC to the candidate epitope, 100 μM of biotinylated epitope was incubated with variable ratios of radio labeled to unlabeled peptide PanC for 2 hours in a volume of 1504, of PBS pH 7.2. After 2 hours of incubation, the mixture was combined with Neutravidin beads (Pierce, Rockford Ill.) to immobilize biotin conjugated epitope and any peptide PanC that may be bound to it. Beads were washed 3× with PBS to remove unbound peptide. Beads were subsequently counted for bound radioactive peptide.

Binding Competition Between Epitope of Hsc71 and Hsp70

A competition binding study was performed in order to demonstrate that binding of PanC to various HSP70 family members is mediated by binding of PanC to the epitope GIPPAPRGVPQIEVTF (SEQ ID NO:15). A 96 well plate was prepared by coating each well with Hsp70 (BioVision, Mountain View, Calif.) or BSA (control) by adding 0.5 μg/well in 1004 of PBS and incubating overnight at 4C. The next day, each well was washed 3× with PBS. 99m-Tc labeled PanC, 0.1 μCi, and various concentrations of the synthetic epitope were added in a volume of 1504 of PBS, pH 7.4, to each well and incubated for 1 hour at room temperature. At the end of incubation, each well was washed 3× with PBS. Bound PanC was eluted from each well in glycine tris HCl buffer, pH 2.8, and counted in a gamma counter to determine fractional binding.

### Example 4

### Use of Chimeric Antibody in Cancer Treatment

Antibodies against the epitope GIPPAPRGVPQIEVTF (SEQ ID NO:15) can be prepared using the methods described herein or known in the art. Additional detailed protocols are available in, e.g., Short Protocols in Molecular Biology, 5^{th} ed., by Ausubel et al., or in Antibodies: A Laboratory Manual, by Harlow and Lane. An antibody of the invention can be further modified to include one or more of the therapeutic or cytotoxic agents described herein, and can be tested for its ability to treat cancer in a patient in need thereof as follows.

Fifteen patients with stage IIIB or stage IV nonsmall cell lung cancer will be enrolled in a phase I clinical trial. Patients will receive a once per week intravenous infusion of 125 mg/m2, 250 mg/m2, or 375 mg/m2 of the antibody for four weeks. Patients will be monitored for infusion related side effects. Complete blood counts, liver function tests, and metabolic profiles will be monitored weekly for six months. Tumor response will be assessed using PET and CT imaging to compare tumor size and metabolism at 0, 1, 2, 3, and 6 months study enrollment time. Survival will be monitored with Kaplan-Meier analysis for twelve months.

### Other Embodiments

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

What is claimed is:

1-85. (canceled)

86. A method of diagnosing or imaging cancer in a human, said method comprising
(a) administering to said human an agent comprising contiguous amino acids having the sequence set forth in any one of SEQ ID NOs:1-14 and a detectable label; and
(b) diagnosing or imaging said cancer in said human by detecting binding of said agent to a cell of said human.

87. The method of claim 86, wherein said cancer is prostate cancer, colon cancer, lung cancer, or breast cancer.

88. The method of claim 86, wherein said agent is a peptide agent.

89. The method of claims 86, wherein said agent is an antibody comprising said amino acids.

90. The method of claim 86 further comprising, after step (a), allowing said agent to bind to cells in the body of said human and allowing said agent that remains unbound to be cleared from the body of said human; and obtaining an image of a region of the body of said human comprising said cancer.

91. An agent comprising contiguous amino acids having the sequence set forth in any one of SEQ ID NOs: 1 and 3-14.

92. The agent of claim 91, wherein said agent binds to an amino acid epitope having at least 90% sequence identity to the sequence set forth in SEQ ID NO:15.

93. The agent of claim 91, further comprising one or more of a detectable label, a therapeutic agent, a chelating agent, or a linking agent.

94. The agent of claim 93, wherein said therapeutic agent is a cytotoxic agent.

95. The agent of claim 91, wherein said agent is a peptide agent.

96. The agent of claim 91, wherein said agent is an antibody comprising said amino acids.

97. A method of treating cancer in a human, said method comprising administering to said human an agent comprising contiguous amino acids having the sequence set forth in any one of SEQ ID NOs:1-14 and a cytotoxic agent.

98. The method of claim 97, wherein said agent is an antibody comprising said amino acids.

99. A method of treating cancer in a human, said method comprising administering to said human a nucleic acid molecule encoding contiguous amino acids having the sequence set forth in any one of SEQ ID NOs:1-14 and a cytotoxic agent.

100. The method of claim 99, wherein said nucleic acid molecule encodes an antibody comprising said amino acids.

101. A method of making an antibody, comprising
(a) recombinantly expressing a nucleic acid molecule that encodes contiguous amino acids having the sequence set forth in one or more of SEQ ID NOs:1-14, wherein said amino acids specifically bind to an epitope having at least 90% sequence identity to the sequence set forth in SEQ ID NO:15.

102. A method of making an antibody, comprising:
(a) injecting a mammal with a peptide comprising an amino acid sequence having at least 90% sequence identity to the sequence set forth in SEQ ID NO:15;
(b) collecting ascites from said mammal; and
(c) purifying said antibody from said ascites, wherein said antibody specifically binds said peptide.

103. A method of identifying a small molecule capable of specifically binding to a cancer cell comprising:
(a) contacting said small molecule to a peptide, polypeptide, phage, or fusion molecule comprising an amino acid sequence having at least 90% sequence identity to the sequence set forth in SEQ ID NO:15; and
(b) determining the binding affinity of said small molecule to said peptide, polypeptide, phage, or fusion molecule, wherein the determination that said small molecule binds to said peptide, polypeptide, phage, or fusion molecule with a dissociation constant of less than 10⁻⁷M indicates said small molecule is capable of specifically binding said cancer cell.

104. A method of identifying a small molecule capable of specifically binding to a cancer cell comprising contacting the small molecule identified by the method of claim 103 to a cancer cell, whereby the binding of said small molecule to said cancer cell but not to a non-cancerous cell indicates said small molecule is capable of specifically binding to said cancer cell.


SEQUENCE LISTING (15 sequences)

SEQ ID NO: 1 - protein, 7 aa, Homo sapiens
KIFVWPY

SEQ ID NO: 2 - protein, 7 aa, Homo sapiens
KLWVIPQ

SEQ ID NO: 3 - protein, 7 aa, Homo sapiens
QLWVQPL

SEQ ID NO: 4 - protein, 7 aa, Homo sapiens
TFSTLVW

SEQ ID NO: 5 - protein, 7 aa, Homo sapiens
KVWTIPR

SEQ ID NO: 6 - protein, 7 aa, Homo sapiens
MPPQFYN

SEQ ID NO: 7 - protein, 12 aa, Homo sapiens
DYWDTSWPLLLF

SEQ ID NO: 8 - protein, 12 aa, Homo sapiens
LCTRAWCYQSPT

SEQ ID NO: 9 - protein, 12 aa, Homo sapiens
APWHLSSQVSRT

SEQ ID NO: 10 - protein, 9 aa, Homo sapiens
CPGTPWNQC

SEQ ID NO: 11 - protein, 12 aa, Homo sapien
DYWDTSWPLLLF

SEQ ID NO: 12 - protein, 17 aa, Homo sapien
DYWDTSWPLLLFGGGSK

SEQ ID NO: 13 - protein, 37 aa, Homo sapien
DYWDTSWPLLLFGGGSKFAKFAKKFAKFAKKFAKFAK

SEQ ID NO: 14 - protein, 16 aa, Homo sapien
DYWDTSWPLLLFGGGC

SEQ ID NO: 15 - protein, 16 aa, Homo sapien
GIPPAPRGVPQIEVTF